SEQUENCE LISTING (20 sequences)

SEQ ID NO: 1 - DNA, 2178 bp, Arabidopsis sp.
atggagattaacggggcacacaagagcaacggaggaggagtggacgctatgttatgcggcggagacatcaagacaaagaacatggtgatcaacgcggaggatcctctcaactggggagctgcagcggagcaaatgaaaggtagccatttggatgaagtgaagagaatggttgctgagtttaggaagccagttgtgaatcttggtggtgagactctgaccattggacaagtggctgcgatctcaactattggtaacagtgtgaaggtggagctatcggagacagctagagccggtgtgaatgctagtagtgattgggttatggagagtatgaacaaaggcactgatagttatggtgttactactggttttggtgctacttctcatcggagaaccaaaaacggtgtcgcacttcagaaggaacttattagattccttaacgccggaatattcggaagcacgaaagaaacaagccacacattgccacactccgccacaagagccgccatgcttgtacgaatcaacactctcctccaaggattttccggtatccgatttgagattctcgaagcaattaccagtttcctcaacaacaacatcactccatctctccccctccgtggtacaatcaccgcctccggagatctcgttcctctctcctacatcgccggacttctcaccggtcgtcccaattccaaagctactggtcccaacggtgaagctttaacagcagaggaagctttcaaattagcaggaatcagctccggattctttgatctccagcctaaggaaggtctcgcgctagtcaatggcacggcggttggatctggaatggcgtcaatggtgttattcgaaacgaatgttctctctgttttggctgagattttgtcggcggttttcgcagaggtgatgagtggtaagcctgagttcaccgatcatctcactcacagacttaaacatcatcccggtcaaatcgaagcggcggcgataatggagcatatcctcgacggaagctcgtacatgaaattagctcagaagcttcacgagatggatccgttacagaaacctaaacaagatcgttacgctcttcgtacttctcctcaatggttaggtcctcaaatcgaagtgatccgttacgcaacgaaatcgatcgagcgtgagattaactccgtcaacgataatccgttgatcgatgtttcgaggaacaaggcgattcacggtggtaacttccaaggaacaccaatcggagtttcaatggataacacgagattggcgatagcagcgattggtaaactcatgtttgctcaattctcagagcttgtgaatgatttctacaacaatggtttaccctcgaatctaaccgcttcgaggaatccaagtttggattatggattcaagggagctgagattgcaatggcttcttattgttcagagcttcaatacttagctaatcctgtgactagccatgttcaatcagcagagcaacataaccaagatgtcaactctttgggactaatctcgtctcgcaaaacttctgaagctgttgatattctcaagcttatgtcaacaacgttcctcgttgcgatttgtcaagctgtggatttgagacatttggaggagaatttgagacagactgtgaagaacactgtctctcaagtggcgaagaaagttcttactactggagtcaatggtgagcttcatccttctcgcttctgcgaaaaggatttactcaaagttgtagaccgtgaacaagtctacacatacgcggatgatccttgtagcgcaacgtacccgttgattcagaagctgagacaagttattgttgaccatgctttgatcaatggtgagagtgagaagaatgcagtgacttcaatcttccataagattggagctttcgaggaggagcttaaggcagtgctaccgaaagaagtggaagcagcaagagcagcctacgataacggaacatcggctatcccgaacaggatcaaggaatgtaggtcgtatccattgtatagattcgtgagggaagagcttggaacagagcttttgaccggagagaaagtgacgtcgcctggagaagagttcgacaaggttttcacggcgatttgtgaaggtaaaatcattgatccgatgatggaatgtctcaacgagtggaacggagctcccattccaatatgttaa

SEQ ID NO: 2 - protein, 725 aa, Arabidopsis sp.
MEINGAHKSNGGGVDAMLCGGDIKTKNMVINAEDPLNWGAAAEQMKGSHLDEVKRMVAEFRKPVVNLGGETLTIGQVAAISTIGNSVKVELSETARAGVNASSDWVMESMNKGTDSYGVTTGFGATSHRRTKNGVALQKELIRFLNAGIFGSTKETSHTLPHSATRAAMLVRINTLLQGFSGIRFEILEAITSFLNNNITPSLPLRGTITASGDLVPLSYIAGLLTGRPNSKATGPNGEALTAEEAFKLAGISSGFFDLQPKEGLALVNGTAVGSGMASMVLFETNVLSVLAEILSAVFAEVMSGKPEFTDHLTHRLKHHPGQIEAAAIMEHILDGSSYMKLAQKLHEMDPLQKPKQDRYALRTSPQWLGPQIEVIRYATKSIEREINSVNDNPLIDVSRNKAIHGGNFQGTPIGVSMDNTRLAIAAIGKLMFAQFSELVNDFYNNGLPSNLTASRNPSLDYGFKGAEIAMASYCSELQYLANPVTSHVQSAEQHNQDVNSLGLISSRKTSEAVDILKLMSTTFLVAICQAVDLRHLEENLRQTVKNTVSQVAKKVLTTGVNGELHPSRFCEKDLLKVVDREQVYTYADDPCSATYPLIQKLRQVIVDHALINGESEKNAVTSIFHKIGAFEEELKAVLPKEVEAARAAYDNGTSAIPNRIKECRSYPLYRFVREELGTELLTGEKVTSPGEEFDKVFTAICEGKIIDPMMECLNEWNGAPIPIC

SEQ ID NO: 3 - DNA, 2154 bp, Arabidopsis sp.
atggatcaaatcgaagcaatgttgtgcggcggaggagagaagacaaaagtggcggttactacgaagactttggcagatccattgaattggggtttagcagcggatcaaatgaaaggaagtcatttagatgaagtgaagaagatggtcgaagagtatcgtagaccagtcgtgaatcttggcggagaaacactgacgatcggacaagttgctgccatctccaccgtaggaggcagcgttaaggttgagttagcggagacttcaagagccggtgtgaaagctagcagtgattgggttatggagagcatgaacaaaggtactgacagttacggagtcaccaccggctttggtgctacttctcaccggagaaccaaaaacggcaccgcattacaaacagaactcattagatttttgaacgccggaatattcggaaacacgaaggagacatgtcacacactgccgcaatccgccacaagagccgccatgctcgtcagagtcaacactcttctccaaggatactccgggatccgattcgagatcctcgaagcgattacaagtctcctcaaccacaacatctctccgtcactacctctccgtggaaccattaccgcctccggcgatctcgttcctctctcttacatcgccggacttctcaccggccgtcctaattccaaagccaccggtcccgacggtgaatcgctaaccgcgaaagaagcttttgagaaagccggaatcagtactggattcttcgatttacaacctaaggaaggtttagctctcgttaatggcacggcggttggatctggaatggcgtcgatggttctattcgaagcgaatgtccaagcggtgttagcggaggttttatcagcgatcttcgcggaggttatgagcgggaaacctgagtttaccgatcatctgactcatcgtttaaaacatcatcccggacaaatcgaagcggcggcgataatggagcacatactcgacggaagctcatacatgaaattagctcaaaaggttcacgagatggatccattgcagaaaccaaaacaagatcgttacgctcttcgtacatctcctcaatggctaggtcctcaaattgaagtaatccgtcaagctacgaaatcgatagagcgtgaaatcaactccgttaacgataatccgttgatcgatgtttcgaggaacaaggcgattcacggtggtaacttccaaggaacaccaatcggagtttctatggataacacgagattggcgattgctgcgattgggaagctaatgtttgctcaattctctgagcttgttaatgatttctacaacaatggacttccttcgaatctaactgcttcgagtaatccaagtttggattatggattcaaaggagcagagattgctatggcttcttattgttctgagcttcaatacttggctaatccagtcacaagccatgttcaatcagctgagcaacataatcaagatgtgaactctcttggtttgatctcgtctcgtaaaacatctgaagctgtggatattcttaagctaatgtcaacaacgttccttgtggggatatgtcaagctgttgatttgagacatttggaggagaatctgagacaaactgtgaagaacacagtttctcaagttgctaagaaagtgttaaccactggaatcaacggtgagttacatccgtcaaggttttgcgagaaggacttgcttaaggttgttgatcgtgagcaagtgttcacgtatgtggatgatccttgtagcgctacgtacccgttgatgcagagactaagacaagttattgttgatcacgctttgtccaacggtgagactgagaagaatgcagtgacttcgatctttcaaaagattggagcttttgaagaggagcttaaggctgtgcttccaaaggaagttgaagcggctagagcggcttatgggaatggaactgcgccgattcctaaccggattaaggaatgtaggtcgtatccgttgtataggttcgtgagggaagagcttggaacgaagttgttgactggagaaaaggttgtgtctccgggagaggagtttgataaggtcttcactgctatgtgtgaaggtaaacttattgatccgttgatggattgtctcaaggaatggaacggagctccgattccgatttgctaa

SEQ ID NO: 4 - protein, 717 aa, Arabidopsis sp.
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 5 - DNA, 2124 bp, Arabidopsis sp.
atggagctatgcaatcaaaacaatcacatcaccgccgtctcgggcgatccgttgaactggaacgcgacggccgaagctttgaaagggagccacctggatgaggtgaaacgaatggtgaaagagtataggaaagaggcggtgaagttaggaggtgagactttgacgattggtcaagtagccgccgtggctagaggaggaggaggatctacggtggagctagcggaggaggctcgtgccggagtcaaggcgagtagcgaatgggtgatggagagcatgaaccgaggaacggacagttatggagttaccacagggtttggtgcaacttcccatagaagaaccaaacaaggcggtgcacttcaaaatgagcttattaggttcttgaatgccggaatatttggccccggcgccggggacacgtcacacacgttgccaaagccgacaacaagagcggcaatgctcgtccgtgtcaacactctcctccaaggctactccggtatacgcttcgagattctcgaagcaattacaaagcttctcaaccacgaaatcactccgtgcctccctctccgtggcaccatcaccgcctccggtgaccttgttcctctctcttacatcgccggacttctcactggccgtcccaactccaaagccgtgggtccctctggtgagactctcactgcctctgaggcctttaagctcgccggagtatcgtcctttttcgagctgcagcctaaggaaggactagcacttgtgaacgggacagcggttggatcgggtttggcctcaacggttttgttcgatgcaaatattttggctgttttatcggaagttatgtctgccatgttcgcagaggttatgcaagggaaaccggagtttacagatcatcttacgcataagctcaagcaccatcccggtcagatcgaagccgccgcaattatggaacatatattagacggaagctcttacgttaaagaagctcaacttctccacgaaatggatcctcttcaaaaacctaaacaagatcggtacgctttacgtacgtcaccacaatggcttgggccgcagattgaagtgatcagagcggctactaaaatgattgagcgtgagatcaactctgttaatgataaccctttgatagatgtgtcgaggaacaaggcgttgcacggtggaaatttccaagggacaccgatcggtgttgccatggataattcccgtctagccattgcttccattgggaaactcatgtttgcgcagttttctgaactagtgaacgatttctacaacaatggtttgccttctaatctatctggtgggagaaaccctagtcttgattacgggtttaaaggcgcggaaatagccatggcttcttattgctccgagcttcagttcctggctaatcccgtgaccaaccatgtccaaagcgcagagcagcataaccaagacgttaattccctagggctaatctctagcaggaaaactgcagaagcagtggatatcctcaagctaatgtccacaacctacttagtcgcgctttgccaagccgttgatctaagacatcttgaagagaatctgaagaaggcggttaaatcagcagtgagtcaggtggcgaaacgggtcttaaccgttggtgccaacggggagctacatccgtcaaggttcacagaacgtgatgtcctccaagtggttgaccgagagtacgtgttctcatacgcagacgatccctgcagcctcacttacccgctaatgcagaaacttagacacattcttgtagaccacgctttagcggatccagaacgcgaggccaattccgcgacatcggttttccacaaaatcggagcttttgaagccgagctgaaactgcttctccctaaagaagtagaacgcgtccgggttgaatacgaggaaggaacatcggctatagctaaccggattaaggaatgtcggtcttatccattgtatcggtttgtccgcgatgagctaaatactgaactgcttactggagagaatgttcggtcgccaggagaggagtttgataaagtgttcttagcgatttctgatggaaaacttattgatccgttgttggaatgtctcaaggagtggaacggagctccggtttcaatctgttga

SEQ ID NO: 6 - protein, 707 aa, Arabidopsis sp.
MELCNQNNHITAVSGDPLNWNATAEALKGSHLDEVKRMVKEYRKEAVKLGGETLTIGQVAAVARGGGGSTVELAEEARAGVKASSEWVMESMNRGTDSYGVTTGFGATSHRRTKQGGALQNELIRFLNAGIFGPGAGDTSHTLPKPTTRAAMLVRVNTLLQGYSGIRFEILEAITKLLNHEITPCLPLRGTITASGDLVPLSYIAGLLTGRPNSKAVGPSGETLTASEAFKLAGVSSFFELQPKEGLALVNGTAVGSGLASTVLFDANILAVLSEVMSAMFAEVMQGKPEFTDHLTHKLKHHPGQIEAAAIMEHILDGSSYVKEAQLLHEMDPLQKPKQDRYALRTSPQWLGPQIEVIRAATKMIEREINSVNDNPLIDVSRNKALHGGNFQGTPIGVAMDNSRLAIASIGKLMFAQFSELVNDFYNNGLPSNLSGGRNPSLDYGFKGAEIAMASYCSELQFLANPVTNHVQSAEQHNQDVNSLGLISSRKTAEAVDILKLMSTTYLVALCQAVDLRHLEENLKKAVKSAVSQVAKRVLTVGANGELHPSRFTERDVLQVVDREYVFSYADDPCSLTYPLMQKLRHILVDHALADPEREANSATSVFHKIGAFEAELKLLLPKEVERVRVEYEEGTSAIANRIKECRSYPLYRFVRDELNTELLTGENVRSPGEEFDKVFLAISDGKLIDPLLECLKEWNGAPVSIC

SEQ ID NO: 7 - DNA, 1512 bp, Saccharomyces cerevisiae
atgaggaagctaaatccagctttagaatttagagactttatccaggtcttaaaagatgaagatgacttaatcgaaattaccgaagagattgatccaaatctcgaagtaggtgcaattatgaggaaggcctatgaatcccacttaccagccccgttatttaaaaatctcaaaggtgcttcgaaggatcttttcagcattttaggttgcccagccggtttgagaagtaaggagaaaggagatcatggtagaattgcccatcatctggggctcgacccaaaaacaactatcaaggaaatcatagattatttgctggagtgtaaggagaaggaacctctccccccaatcactgttcctgtgtcatctgcaccttgtaaaacacatatactttctgaagaaaaaatacatctacaaagcctgccaacaccatatctacatgtttcagacggtggcaagtacttacaaacgtacggaatgtggattcttcaaactccagataaaaaatggactaattggtcaattgctagaggtatggttgtagatgacaagcatatcactggtctggtaattaaaccacaacatattagacaaattgctgactcttgggcagcaattggaaaagcaaatgaaattcctttcgcgttatgttttggcgttcccccagcagctattttagttagttccatgccaattcctgaaggtgtttctgaatcggattatgttggcgcaatcttgggtgagtcggttccagtagtaaaatgtgagaccaacgatttaatggttcctgcaacgagtgagatggtatttgagggtactttgtccttaacagatacacatctggaaggcccatttggtgagatgcatggatatgttttcaaaagccaaggtcatccttgtccattgtacactgtcaaggctatgagttacagagacaatgctattctacctgtttcgaaccccggtctttgtacggatgagacacataccttgattggttcactagtggctactgaggccaaggagctggctattgaatctggcttgccaattctggatgcctttatgccttatgaggctcaggctctttggcttatcttaaaggtggatttgaaagggctgcaagcattgaagacaacgcctgaagaattttgtaagaaggtaggtgatatttactttaggacaaaagttggttttatagtccatgaaataattttggtggcagatgatatcgacatatttaacttcaaagaagtcatctgggcctacgttacaagacatacacctgttgcagatcagatggcttttgatgatgtcacttcttttcctttggctccctttgtttcgcagtcatccagaagtaagactatgaaaggtggaaagtgcgttactaattgcatatttagacagcaatatgagcgcagttttgactacataacttgtaattttgaaaagggatatccaaaaggattagttgacaaagtaaatgaaaattggaaaaggtacggatataaataa

SEQ ID NO: 8 - protein, 503 aa, Saccharomyces cerevisiae
MRKLNPALEFRDFIQVLKDEDDLIEITEEIDPNLEVGAIMRKAYESHLPAPLFKNLKGASKDLFSILGCPAGLRSKEKGDHGRIAHHLGLDPKTTIKEIIDYLLECKEKEPLPPITVPVSSAPCKTHILSEEKIHLQSLPTPYLHVSDGGKYLQTYGMWILQTPDKKWTNWSIARGMVVDDKHITGLVIKPQHIRQIADSWAAIGKANEIPFALCFGVPPAAILVSSMPIPEGVSESDYVGAILGESVPVVKCETNDLMVPATSEMVFEGTLSLTDTHLEGPFGEMHGYVFKSQGHPCPLYTVKAMSYRDNAILPVSNPGLCTDETHTLIGSLVATEAKELAIESGLPILDAFMPYEAQALWLILKVDLKGLQALKTTPEEFCKKVGDIYFRTKVGFIVHEIILVADDIDIFNFKEVIWAYVTRHTPVADQMAFDDVTSFPLAPFVSQSSRSKTMKGGKCVTNCIFRQQYERSFDYITCNFEKGYPKGLVDKVNENWKRYGYK

SEQ ID NO: 9 - DNA, 729 bp, Saccharomyces cerevisiae
atgctcctatttccaagaagaactaatatagcctttttcaaaacaacaggcatttttgctaattttcctttgctaggtagaaccattacaacttcaccatctttccttacacataaactgtcaaaggaagtaaccagggcatcaacttcgcctccaagaccaaagagaattgttgtcgcaattactggtgcgactggtgttgcactgggaatcagacttctacaagtgctaaaagagttgagcgtagaaacccatttggtgatttcaaaatggggtgcagcaacaatgaaatatgaaacagattgggaaccgcatgacgtggcggccttggcaaccaagacatactctgttcgtgatgtttctgcatgcatttcgtccggatctttccagcatgatggtatgattgttgtgccctgttccatgaaatcactagctgctattagaatcggttttacagaggatttaattacaagagctgccgatgtttcgattaaagagaatcgtaagttactactggttactcgggaaacccctttatcttccatccatcttgaaaacatgttgtctttatgcagggcaggtgttataatttttcctccggtacctgcgttttatacaagacccaagagccttcatgacctattagaacaaagtgttggcaggatcctagactgctttggcatccacgctgacacttttcctcgttgggaaggaataaaaagcaagtaa

SEQ ID NO: 10 - protein, 242 aa, Saccharomyces cerevisiae
MLLFPRRTNIAFFKTTGIFANFPLLGRTITTSPSFLTHKLSKEVTRASTSPPRPKRIVVAITGATGVALGIRLLQVLKELSVETHLVISKWGAATMKYETDWEPHDVAALATKTYSVRDVSACISSGSFQHDGMIVVPCSMKSLAAIRIGFTEDLITRAADVSIKENRKLLLVTRETPLSSIHLENMLSLCRAGVIIFPPVPAFYTRPKSLHDLLEQSVGRILDCFGIHADTFPRWEGIKSK

SEQ ID NO: 11 - DNA, 26 bp, Artificial Sequence
  Description of Artificial Sequence Synthetic primer
atgaggaagctaaatccagctttaga

SEQ ID NO: 12 - DNA, 25 bp, Artificial Sequence
  Description of Artificial Sequence Synthetic primer
ttatttatatccgtaccttttccaa

SEQ ID NO: 13 - DNA, 25 bp, Artificial Sequence
  Description of Artificial Sequence Synthetic primer
atgctcctatttccaagaagaacta

SEQ ID NO: 14 - DNA, 25 bp, Artificial Sequence
  Description of Artificial Sequence Synthetic primer
ttacttgctttttattccttcccaa

SEQ ID NO: 15 - DNA, 636 bp, Saccharomyces cerevisiae
tcaccatctttccttacacataaactgtcaaaggaagtaaccagggcatcaacttcgcctccaagaccaaagagaattgttgtcgcaattactggtgcgactggtgttgcactgggaatcagacttctacaagtgctaaaagagttgagcgtagaaacccatttggtgatttcaaaatggggtgcagcaacaatgaaatatgaaacagattgggaaccgcatgacgtggcggccttggcaaccaagacatactctgttcgtgatgtttctgcatgcatttcgtccggatctttccagcatgatggtatgattgttgtgccctgttccatgaaatcactagctgctattagaatcggttttacagaggatttaattacaagagctgccgatgtttcgattaaagagaatcgtaagttactactggttactcgggaaacccctttatcttccatccatcttgaaaacatgttgtctttatgcagggcaggtgttataatttttcctccggtacctgcgttttatacaagacccaagagccttcatgacctattagaacaaagtgttggcaggatcctagactgctttggcatccacgctgacacttttcctcgttgggaaggaataaaaagcaagtaa

SEQ ID NO: 16 - protein, 212 aa, Saccharomyces cerevisiae
MSPSFLTHKLSKEVTRASTSPPRPKRIVVAITGATGVALGIRLLQVLKELSVETHLVISKWGAATMKYETDWEPHDVAALATKTYSVRDVSACISSGSFQHDGMIVVPCSMKSLAAIRIGFTEDLITRAADVSIKENRKLLLVTRETPLSSIHLENMLSLCRAGVIIFPPVPAFYTRPKSLHDLLEQSVGRILDCFGIHADTFPRWEGIKSK

SEQ ID NO: 17 - DNA, 684 bp, Aspergillus niger
atgttcaactcacttctatccggtactactacaccaaactccggccgtgcatctcctcccgccagcgaaatgcccatcgataatgatcacgtggccgttgcccgtccagctccccgccgccgccgcattgtagtagccatgacgggtgccactggagccatgctcggcatcaaagtcctaattgctctgcgccgtctaaatgtggagacacacctggtgatgagtaaatgggcggaggctacgatcaaatacgagactgactaccatccctcaaacgtgcgagcgctggccgactacgtgcacaacatcaatgacatggccgccccagtatccagcggctcattccgcgcggacggaatgattgtggtaccgtgcagcatgaaaacattggctgctatccactcgggcttttgcgacgatctcatttcaaggacagcagatgtgatgctcaaggagcgcaggcggttggtgctagtagcgcgggagacgccattgagcgagatccatctgcgaaacatgttggaggttacacgcgctggggcagtcatcttccccccagtaccggcgttctacatcaaggccggaagtatcgaggacctcatcgaccagagtgttggacgaatgttggatttatttgacctcgacacgggggattttgaacgttggaatggatgggaaaaataa

SEQ ID NO: 18 - protein, 227 aa, Aspergillus niger
MFNSLLSGTTTPNSGRASPPASEMPIDNDHVAVARPAPRRRRIVVAMTGATGAMLGIKVLIALRRLNVETHLVMSKWAEATIKYETDYHPSNVRALADYVHNINDMAAPVSSGSFRADGMIVVPCSMKTLAAIHSGFCDDLISRTADVMLKERRRLVLVARETPLSEIHLRNMLEVTRAGAVIFPPVPAFYIKAGSIEDLIDQSVGRMLDLFDLDTGDFERWNGWEK

SEQ ID NO: 19 - DNA, 1503 bp, Aspergillus niger
atgtctgcgcaacctgctcacctgtgtttccgctccttcgtcgaagccctcaaggtcgacaacgaccttgttgaaatcaataccccaattgaccccaatctcgaagctgctgctattacccgccgagtatgtgagaccaacgacaaggctcctttattcaacaacctcatcggcatgaaaaatggcctcttccgtatacttggggctcctggctctctcaggaagtcgtctgctgatcgctacggccgccttgctcgtcacctagccctcccacctacggcctcaatgcgtgagattctcgataagatgctctccgccagcgatatgcctcccatccctccgaccattgttcccaccgggccatgcaaggagaacagcttagatgactctgaattcgaccttaccgaactccccgttcctcttattcacaaatcggatggtggtaaatacatccaaacctatggcatgcacattgtgcagtctccggatggaacctggaccaactggtctattgcccgtgcgatggtccatgacaagaaccatctgaccggcctggttattccccctcagcacatctggcagattcaccagatgtggaagaaggaaggccgcagtgacgttccctgggctttggcctttggtgtcccacccgctgccattatggcctctagcatgcctattcccgatggtgtcaccgaagctgggtacgtgggagctatgacgggatcctccctggagcttgttaaatgtgatacgaacgatctatatgtccccgctacctcagaaatcgttctcgagggcacactctctatcagcgagacaggcccagagggacctttcggtgagatgcatggttacatcttccccggggatactcacctcggcgccaaatacaaggttaaccggatcacctaccgcaacaacgccatcatgcccatgtcttcttgtggccgcttgacggatgaaacgcacaccatgatcggctctctggctgcggcggagatccgtaagctctgccagcagaatgacctccctatcactgatgccttcgctcctttcgagtctcaagttacctgggttgctctgcgggtcgatactgagaagctacgtgccatgaagacaacgtctgagggattccgcaagagagtgggagacgtcgtcttcaaccacaaggccggatacaccattcatcgtctggtgttggtcggtgacgacattgatgtctatgaaggaaaggatgtgctctgggcgttctccacccgttgccgtcctggtatggacgagactttgtttgaggatgttcgtgggttccccttgattccgtatatgggacacgggaatgggcccgcccaccgcggcggaaaggttgtgtccgacgctcttatgccgactgagtacaccactggtcgcaactgggaggctgctgacttcaaccaatcttatcccgaggatctgaagcagaaggtgttggacaactggacgaagatgggtttcagcaactaa

SEQ ID NO: 20 - protein, 500 aa, Aspergillus niger
MSAQPAHLCFRSFVEALKVDNDLVEINTPIDPNLEAAAITRRVCETNDKAPLFNNLIGMKNGLFRILGAPGSLRKSSADRYGRLARHLALPPTASMREILDKMLSASDMPPIPPTIVPTGPCKENSLDDSEFDLTELPVPLIHKSDGGKYIQTYGMHIVQSPDGTWTNWSIARAMVHDKNHLTGLVIPPQHIWQIHQMWKKEGRSDVPWALAFGVPPAAIMASSMPIPDGVTEAGYVGAMTGSSLELVKCDTNDLYVPATSEIVLEGTLSISETGPEGPFGEMHGYIFPGDTHLGAKYKVNRITYRNNAIMPMSSCGRLTDETHTMIGSLAAAEIRKLCQQNDLPITDAFAPFESQVTWVALRVDTEKLRAMKTTSEGFRKRVGDVVFNHKAGYTIHRLVLVGDDIDVYEGKDVLWAFSTRCRPGMDETLFEDVRGFPLIPYMGHGNGPAHRGGKVVSDALMPTEYTTGRNWEAADFNQSYPEDLKQKVLDNWTKMGFSN

(19) United States
(12) Patent Application Publication
(10) Pub. No.: US 2013/0005012 A1
(43) Pub. Date: Jan. 3, 2013

(54) ENZYMATIC SYSTEM FOR MONOMER SYNTHESIS

(75) Inventor: Xiaodan Yu, Chesterfield, MO (US)

(73) Assignee: PHYTOGENE, INC., Rancho Santa Margarita, CA (US)

(21) Appl. No.: 13/531,470

(22) Filed: Jun. 22, 2012

Related U.S. Application Data
(60) Provisional application No. 61/500,582, filed on Jun. 23, 2011.

(51) Int. Cl. C12P 5/00; C12N 5/10; C12N 1/21; C12N 1/13; C12N 1/19; C12N 1/15

(52) U.S. Cl. 435/166; 435/252.33; 435/254.2; 435/254.11; 435/252.3; 435/257.2; 435/419; 435/252.31; 435/252.35; 435/252.32; 435/252.34; 435/254.21; 435/254.22; 435/254.8; 435/254.23; 435/254.3; 435/415; 435/416; 435/412; 435/414; 435/411; 435/417

(57) ABSTRACT

In vivo methods for production of styrene via a recombinant host cell can include cell expression of at least one polynucleotide encoding a polypeptide having cinnamic acid decarboxylase activity, and at least one polynucleotide encoding a polypeptide having phenylalanine ammonia lyase activity.

### RELATED APPLICATIONS

This application claims the priority benefit of U.S. Provisional Application No. 61/500,582, filed Jun. 23, 2011, the entirety of which is incorporated herein by reference.

### SEQUENCE LISTING

The instant application contains a Sequence Listing which has been submitted in ASCII format via EFS-Web and is hereby incorporated by reference in its entirety. Said ASCII copy, created on Jun. 18, 2012, is named 089516US.txt and is 50,273 bytes in size.

### FIELD

The subject technology relates to methods for microbial production of monomers and, more particularly in some embodiments, to biosynthesis of styrene.

### BACKGROUND

Styrene (vinyl benzene) is an organic compound with a chemical formula of C₈H₈. This cyclic hydrocarbon is a colorless, oily liquid that evaporates easily and has a sweet rubber-like smell. At higher concentrations, styrene confers a less pleasant odor. Styrene is named after the styrax trees (Styrax platanifolius) from which sap (a type of benzoin resin) can be extracted. Low levels of styrene occur naturally in several plant species. A variety of foods such as fruits, vegetables, nuts, beverages, and meats also contains styrene.

Industrially, styrene is the precursor to polystyrene and several copolymers. The presence of the vinyl group allows styrene to polymerize. Approximately 15 billion pounds are produced annually. The production of styrene in the United States increased dramatically during the 1940s, when it was popularized as a feedstock for synthetic rubber. Today, commercially significant products include polystyrene, ABS, styrene-butadiene (SBR) rubber, styrene-butadiene latex, SIS (styrene-isoprene-styrene), S-EB-S (styrene-ethylene/butylene-styrene), styrene-divinylbenzene (S-DVB), styrene-acrylonitrile resin (SAN) and unsaturated polyesters. These materials are used in rubber, plastic, insulation, fiberglass, pipes, automobile and boat parts, food containers, and carpet backing.

Styrene is produced in industrial quantities mostly from ethylbenzene, which is in turn prepared on a large scale by alkylation of benzene with ethylene. It is one of the most important petrochemical products. There are several methods to produce styrene. Dehydrogenation of ethylbenzene is the most common way of production. Ethylbenzene is mixed in the gas phase with 10-15 times its volume in high-temperature steam, and passed over a solid catalyst bed. Most ethylbenzene dehydrogenation catalysts are based on iron(III) oxide, promoted by several percent potassium oxide or potassium carbonate. Steam serves several roles in this reaction. It is the source of heat for powering the endothermic reaction, and it removes coke that tends to form on the iron oxide catalyst through the water gas shift reaction. The potassium promoter enhances this decoking reaction. The steam also dilutes the reactant and products, shifting the position of chemical equilibrium towards products. A typical styrene plant consists of two or three reactors in series, which operate under vacuum to enhance the conversion and selectivity. Typical per-pass conversions are about 65% for two reactors and 70-75% for three reactors. Selectivity to styrene is 93-97%. The main byproducts are benzene and toluene. Because styrene and ethylbenzene have similar boiling points (145 and 136° C., respectively), their separation requires tall distillation towers and high return/reflux ratios. At its distillation temperatures, styrene tends to polymerize. To minimize this problem, early styrene plants added elemental sulfur to inhibit the polymerization. During the 1970s, new free radical inhibitors consisting of nitrated phenol-based retarders were developed. More recently, a number of additives have been developed that exhibit superior inhibition against polymerization.

Since styrene is an essential compound used in many chemical product, alternative production methods, especially the ones that do not require fossil fuels as feed stock, is urgently needed. Hence, despite the availability of some means for producing styrene, there is a continuing need for a new methods for producing styrene monomers that are efficient and less expensive.

### SUMMARY

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 17. The other clauses can be presented in a similar manner.

1. A method for the production of styrene comprising:
(i) contacting a recombinant host cell with a fermentable substrate, said recombinant host comprising:
   a) at least one polynucleotide encoding a polypeptide having phenylalanine ammonia lyase (PAL) activity; and
   b) at least one polynucleotide encoding a polypeptide having cinnamic acid decarboxylase activity; and
(ii) growing said recombinant cell for a time sufficient to produce styrene.

2. The method of clause 1, wherein step (ii) comprises culturing the host cell under a condition in which styrene is produced.

3. The method of clause 1 or 2, wherein said condition comprises at least one of: (i) culturing at a temperature of about 10° C. to about 20° C. or (ii) culturing in the presence of a salt of at least one of calcium, magnesium, sodium, cobalt, molybdenum, potassium, manganese, zinc, copper, or iron.

4. The method of clause 3, wherein the temperature comprises about 16° C.

5. The method of any one of clauses 1-4, wherein the salt comprises at least one of Ca²⁺, Mg²⁺, Mn²⁺, Zn²⁺, or Fe³⁺ ions.

6. The method of any one of clauses 1-5, further comprising recovering said styrene.

7. The method of any one of clauses 1-6, wherein the polypeptide having phenylalanine ammonia lyase (PAL) activity comprises a polypeptide selected from the group consisting of PAL1 as set forth in SEQ ID NO:2, PAL2 as set forth in SEQ ID NO:4, PAL4 as set forth in SEQ ID NO:6, and a functional fragment or variant thereof.

8. The method of any one of clauses 1-7, wherein the polypeptide having cinnamic acid decarboxylase activity comprises a polypeptide selected from the group consisting of ferulic acid decarboxylase (FDC) as set forth in SEQ ID NO:8, AnOHBA1 as set forth in SEQ ID NO:20, and a functional fragment or variant thereof.

9. The method of any one of clauses 1-8, wherein the host cell does not comprise a polynucleotide encoding phenylacrylic acid decarboxylase (PAD1) as set forth in SEQ ID NO:9 or SEQ ID NO:15.

10. The method according to any one of clauses 1-9, wherein said fermentable substrate comprises at least one of a carbon source or a nitrogen source.

11. The method according to clause 10, wherein the carbon source comprises any one of monosaccharides, oligosaccharides, polysaccharides, carbon dioxide, methanol, formaldehyde, formate, glucose, fructose, mannose, galactose, ribose, sorbose, ribulose, lactose, maltose, sucrose, raffinose, starch, cellulose, or carbon-containing amines.

12. The method according to clause 10, wherein the nitrogen source comprises any one of ammonia in liquid or gaseous form, ammonium sulfate, ammonium chloride, ammonium phosphate, ammonium carbonate or ammonium nitrate, nitrates, urea, amino acids, or complex nitrogen sources comprising at least one of corn steep liquor, soya meal, soya protein, yeast extract, or meat extract.

13. The method according to any one of clauses 1-12, wherein said recombinant host cell is selected from the group consisting of bacteria, yeast, filamentous fungi, cyanobacteria algae and plant cells.

14. The method according to any one of clauses 1-12, wherein said recombinant host cell is selected from the group consisting of Escherichia, Salmonella, Bacillus, Acinetobacter, Streptomyces, Corynebacterium, Methylosinus, Methylomonas, Rhodococcus, Pseudomonas, Rhodobacter, Synechocystis; Saccharomyces, Zygosaccharomyces, Kluyveromyces, Candida, Hansenula, Debaryomyces, Mucor, Pichia, Torulopsis, Aspergillus, Arthrobotrys, Brevibacteria, Microbacterium, Arthrobacter, Citrobacter, Escherichia, Klebsiella, Pantoea, Salmonella Corynebacterium, Clostridium, and Clostridium acetobutylicum.

15. The method according to any one of clauses 1-14, wherein said recombinant host cell is a phenylalanine over-producing strain.

16. The method according to any one of clauses 1-12, wherein said recombinant host cell is a cell isolated from plants selected from the group consisting of soybean, rapeseed, sunflower, cotton, corn, tobacco, alfalfa, wheat, barley, oats, sorghum, rice, broccoli, cauliflower, cabbage, parsnips, melons, carrots, celery, parsley, tomatoes, potatoes, strawberries, peanuts, grapes, grass seed crops, sugar beets, sugar cane, beans, peas, rye, flax, hardwood trees, softwood trees, forage grasses, Arabidopsis thaliana, rice (Oryza sativa), switchgrass (Panicum vigratum), Brachypodium spp, Brassica spp., and Crambe abyssinica.

17. The method according to any one of clauses 1-12, wherein the polynucleotide encoding a polypeptide having phenylalanine ammonia lyase activity is derived from Arabidopsis.

18. The method according to any one of clauses 1-12, wherein the polynucleotide encoding a polypeptide having cinnamic acid decarboxylase activity is derived from S. cerevisiae.

19. The method according to any one of clauses 1-18, wherein the styrene can be further converted to other organic molecules by enzymatic or biological conversion within the host cell.

20. The method according to clause 19, wherein the styrene is further converted to toluene, xylene or polystyrene.

21. A recombinant host cell comprising:
at least one polynucleotide encoding a polypeptide having phenylalanine ammonia lyase (PAL) activity; and
at least one polynucleotide encoding a polypeptide having cinnamic acid decarboxylase activity;
wherein said recombinant host cell is capable of producing styrene when grown under a suitable culture condition.

22. The recombinant host cell of clause 21, wherein the polypeptide having phenylalanine ammonia lyase (PAL) activity comprises a polypeptide selected from the group consisting of PAL1 as set forth in SEQ ID NO:2, PAL2 as set forth in SEQ ID NO:4, PAL4 as set forth in SEQ ID NO:6, and a functional fragment or variant thereof.

23. The recombinant host cell of clause 21 or 22, the polypeptide having cinnamic acid decarboxylase activity comprises a polypeptide selected from the group consisting of ferulic acid decarboxylase (FDC) as set forth in SEQ ID NO:8, AnOHBA1 as set forth in SEQ ID NO:20, and a functional fragment or variant thereof.

24. The recombinant host cell of any one of clauses 21-23, wherein the host cell does not comprise a polynucleotide encoding phenylacrylic acid decarboxylase (PAD1) as set forth in SEQ ID NO:9 or SEQ ID NO:15.

25. The method of any one of clauses 21-24, wherein said condition comprises at least one of: (i) at a temperature of about 10° C. to about 20° C. or (ii) culturing in the presence of a salt of at least one of calcium, magnesium, sodium, cobalt, molybdenum, potassium, manganese, zinc, copper or iron.

26. The method of clause 25, wherein the temperature comprises about 16° C.

27. The method of clause 25, wherein the salt comprises at least one of Ca²⁺, Mg²⁺, Mn²⁺, Zn²⁺, Fe³⁺ ions.

28. The method according to any one of clauses 21-27, wherein said suitable culture condition comprises a fermentable substrate comprising at least one of a carbon source or a nitrogen source.

29. The method according to clause 28, wherein the carbon source comprises any one of monosaccharides, oligosaccharides, polysaccharides, carbon dioxide, methanol, formaldehyde, formate, glucose, fructose, mannose, galactose, ribose, sorbose, ribulose, lactose, maltose, sucrose, raffinose, starch or cellulose or carbon-containing amines.

30. The method according to clause 28, wherein the nitrogen source comprises any one of ammonia in liquid or gaseous form, ammonium sulfate, ammonium chloride, ammonium phosphate, ammonium carbonate or ammonium nitrate, nitrates, urea, amino acids, or complex nitrogen sources comprising at least one of corn steep liquor, soya meal, soya protein, yeast extract, or meat extract.

31. A method for the production of styrene comprising:
(i) contacting a recombinant host cell with a fermentable substrate, said recombinant host comprising:
   a) at least one polynucleotide encoding a polypeptide having phenylalanine ammonia lyase (PAL) activity; and
   b) at least one polynucleotide encoding a polypeptide having cinnamic acid decarboxylase activity; and
(ii) growing said recombinant cell for a time sufficient to produce styrene;
wherein the polypeptide having phenylalanine ammonia lyase (PAL) activity comprises a polypeptide of any one of PAL1 as set forth in SEQ ID NO:2, PAL2 as set forth in SEQ ID NO:4, PAL4 as set forth in SEQ ID NO:6, or a functional fragment or variant thereof;
wherein the polypeptide having cinnamic acid decarboxylase activity comprises a polypeptide of any one of ferulic acid decarboxylase (FDC) as set forth in SEQ ID NO:8, AnOHBA1 as set forth in SEQ ID NO:20, or a functional fragment or variant thereof.

32. The method of clause 31, further comprising recovering said styrene.

33. The method of clause 31 or 32, wherein the host cell does not comprise a polynucleotide encoding phenylacrylic acid decarboxylase (PAD1) as set forth in SEQ ID NO:9 or SEQ ID NO:15.

34. The method according to any one of clauses 31-33, wherein said fermentable substrate at least one of a carbon source or a nitrogen source.

35. The method according to any one of clauses 31-34, wherein said recombinant host cell is selected from the group consisting of bacteria, yeast, filamentous fungi, cyanobacteria algae and plant cells.

36. The method according to any one of clauses 31-34, wherein said recombinant host cell is selected from the group consisting of Escherichia, Salmonella, Bacillus, Acinetobacter, Streptomyces, Corynebacterium, Methylosinus, Methylomonas, Rhodococcus, Pseudomonas, Rhodobacter, Synechocystis; Saccharomyces, Zygosaccharomyces, Kluyveromyces, Candida, Hansenula, Debaryomyces, Mucor, Pichia, Torulopsis, Aspergillus, Arthrobotrys, Brevibacteria, Microbacterium, Arthrobacter, Citrobacter, Escherichia, Klebsiella, Pantoea, Salmonella Corynebacterium, Clostridium, and Clostridium acetobutylicum.

37. The method according to any one of clauses 31-36, wherein said recombinant host cell is a phenylalanine over-producing strain.

38. The method according to any one of clauses 31-34, wherein said recombinant host cell is a cell isolated from plants selected from the group consisting of soybean, rapeseed, sunflower, cotton, corn, tobacco, alfalfa, wheat, barley, oats, sorghum, rice, broccoli, cauliflower, cabbage, parsnips, melons, carrots, celery, parsley, tomatoes, potatoes, strawberries, peanuts, grapes, grass seed crops, sugar beets, sugar cane, beans, peas, rye, flax, hardwood trees, softwood trees, forage grasses, Arabidopsis thaliana, rice (Oryza sativa), switchgrass (Panicum vigratum), Brachypodium spp, Brassica spp., and Crambe abyssinica.

39. The method according to any one of clauses 31-34, wherein the polynucleotide encoding a polypeptide having phenylalanine ammonia lyase activity is derived from Arabidopsis.

40. The method according to any one of clauses 31-34, wherein the polynucleotide encoding a polypeptide having cinnamic acid decarboxylase activity is derived from S. cerevisiae.

41. The method according to any one of clauses 31-40, wherein the styrene can be further converted to other organic molecules by enzymatic or biological conversion within the host cell.

42. The method according to clause 41, wherein the styrene is further converted to toluene, xylene or polystyrene.

43. The method according to any one of clauses 31-42, further comprising culturing the host cell under a condition in which styrene is produced.

44. The method according to clause 43, wherein said condition comprises at least one of: (i) at a temperature of about 16° C. or (ii) culturing in the presence of at least one of Ca²⁺, Mg²⁺, Zn²⁺, Fe³⁺ ions.

45. A recombinant host cell comprising:
at least one polynucleotide encoding a polypeptide having phenylalanine ammonia lyase (PAL) activity; and
at least one polynucleotide encoding a polypeptide having cinnamic acid decarboxylase activity;
wherein said recombinant cell produces styrene when grown under a suitable culture condition;
wherein the polypeptide having phenylalanine ammonia lyase (PAL) activity comprises a polypeptide of any one of PAL1 as set forth in SEQ ID NO:2, PAL2 as set forth in SEQ ID NO:4, PAL4 as set forth in SEQ ID NO:6, or a functional fragment or variant thereof;
   wherein the polypeptide having cinnamic acid decarboxylase activity comprises a polypeptide of any one of ferulic acid decarboxylase (FDC) as set forth in SEQ ID NO:8, AnOHBA1 as set forth in SEQ ID NO:20, or a functional fragment or variant thereof.

46. The recombinant host cell of clause 45, wherein the host cell does not contain a polynucleotide encoding phenylacrylic acid decarboxylase (PAD1) as set forth in SEQ ID NO:9 or SEQ ID NO:15.

In an embodiment, the subject technology comprises a recombinant host cell expressing at least one polynucleotide encoding a polypeptide or gene having phenylalanine ammonia lyase activity (e.g., PAL1, PAL2 or PAL4 of Arabidopsis thaliana) and at least one polynucleotide or gene encoding a polypeptide having cinnamic acid decarboxylase activity (e.g., FDC also called as FDC1 of Saccharomyces cerevisiae or AnOHBA1 of Aspergillus niger). In an aspect relating to this embodiment, the host cell does not contain a phenylacrylic acid decarboxylase (PAD1 of Saccharomyces cerevisiae) gene or a polynucleotide encoding a PAD1 polypeptide.

In an embodiment, PAL1 comprises a polypeptide encoded by a nucleic acid sequence shown in SEQ ID NO:1, or comprises a polypeptide sequence shown in SEQ ID NO:2. In an embodiment, PAL2 comprises a polypeptide encoded by a nucleic acid sequence shown in SEQ ID NO:3, or a polypeptide sequence shown in SEQ ID NO:4. In an embodiment, PAL4 comprises a polypeptide encoded by a nucleic acid sequence shown in SEQ ID NO:5, or a polypeptide sequence shown in SEQ ID NO:6. In an embodiment, FDC1 comprises a polypeptide encoded by a nucleic acid sequence shown in SEQ ID NO:7, or a polypeptide sequence shown in SEQ ID NO:8. In an embodiment, AnOHBA1 comprises a polypeptide encoded by a nucleic acid sequence shown in SEQ ID NO:19, or a polypeptide sequence shown in SEQ ID NO:20. In an embodiment, PAD1 comprises a polypeptide encoded by a nucleic acid sequence shown in SEQ ID NO:9, or a polypeptide sequence shown in SEQ ID NO:10.

In some embodiments, the subject technology provides a method for production of styrene comprising: (i) contacting a recombinant host cell with a fermentable carbon substrate, said recombinant host comprising: a) at least one polynucleotide encoding a polypeptide having phenylalanine ammonia lyase activity; and b) at least one polynucleotide encoding a polypeptide having cinnamic acid decarboxylase activity; (ii) growing said recombinant cell for a time sufficient to produce styrene; and (iii) optionally recovering said styrene.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE SEQUENCE LISTING

SEQ ID NO:1 is the nucleic acid sequence of a phenylalanine ammonia lyase 1 (PAL1) enzyme from Arabidopsis.

SEQ ID NO:2 is the polypeptide sequence of PAL1 encoded by SEQ ID NO:1.

SEQ ID NO:3 is the nucleic acid sequence of a phenylalanine ammonia lyase 2 (PAL2) enzyme from Arabidopsis.

SEQ ID NO:4 is the polypeptide sequence of PAL2 encoded by SEQ ID NO:3.

SEQ ID NO:5 is the nucleic acid sequence of a phenylalanine ammonia lyase 4 (PAL4) enzyme from Arabidopsis.

SEQ ID NO:6 is the polypeptide sequence of PAL4 encoded by SEQ ID NO:5.

SEQ ID NO:7 is the nucleic acid sequence of ferulic acid decarboxylase (FDC1/YDR539W) from Saccharomyces cerevisiae.

SEQ ID NO:8 is the polypeptide sequence of FDC1 encoded by SEQ ID NO:7.

SEQ ID NO:9 is the nucleic acid sequence of phenylacrylic acid decarboxylase (PAD1/YDR539W) from S. cerevisiae.

SEQ ID NO:10 is the polypeptide sequence of PAD1 encoded by SEQ ID NO:9.

SEQ ID NO:11 is a forward primer used to amplify FDC1 from genomic DNA of S. cerevisiae.

SEQ ID NO:12 is a reverse primer used to amplify FDC1 from genomic DNA of S. cerevisiae.

SEQ ID NO:13 is a forward primer used to amplify PAD1 from genomic DNA of S. cerevisiae.

SEQ ID NO:14 is a reverse primer used to amplify PAD1 from genomic DNA of S. cerevisiae.

SEQ ID NO:15 is the nucleotide sequence of a truncated phenylacrylic acid decarboxylase (PAD1) from S. cerevisiae.

SEQ ID NO:16 is the polypeptide sequence of PAD1 encoded by SEQ ID NO:15.

SEQ ID NO:17 is the nucleic acid sequence of Aspergillus niger's PADA1 gene (AnPADA1, An03g06570).

SEQ ID NO:18 is the polypeptide sequence of Aspergillus niger's PADA1 (AnPADA1) encoded by SEQ ID NO:17.

SEQ ID NO:19 is the nucleic acid sequence of Aspergillus niger's OHBA1 gene (AnOHBA1, An03g06590).

SEQ ID NO:20 is the polypeptide sequence of Aspergillus niger's OHBA1 encoded by SEQ ID NO:19 (AnOHBA1).

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 is an HPLC profile of the products by yeast expressing PAD1 and feeding on cinnamic acid. Peak 1 is cinnamic acid.

FIG. 2 is an HPLC profile of the products by Escherichia coli expressing PAD1 and feeding on cinnamic acid. Peak 1 is cinnamic acid; peak 2 is an unknown compound in the E. coli culture.

FIG. 3 is an HPLC profile of the products by E. coli expressing a truncated PAD1 (−TP, or ΔPAD1) and feeding on cinnamic acid. Peak 1 is cinnamic acid; peak 2 is an unknown compound in the E. coli culture.

FIG. 4 is an HPLC profile of the products by yeast expressing FDC1 and feeding on cinnamic acid. Peak 1 is cinnamic acid; peak 2 is styrene.

FIG. 5 is an HPLC profile of the products by yeast expressing FDC1 and feeding on p-coumaric acid. Peak 1 is p-coumaric acid; peak 2 is 4-hydroxystyrene.

FIG. 6 is an HPLC profile of the products by yeast expressing FDC1 and feeding on ferulic acid. Peak 1 is ferulic acid; peak 2 is 4-vinyl guaiacol (4-VG).

FIG. 7 is an HPLC profile of the products by E. coli overexpressing yeast FDC1 polynucleotide and feeding on cinnamic acid. Peak 1 is cinnamic acid; peak 2 is an unknown E. coli compound; and peak 3 is styrene.

FIG. 8 is an HPLC profile of the products by E. coli over-expressing FDC and feeding on ferulic acid. Peak 1 is ferulic acid; peak 2 is 4-VG; peak 3 is an unknown E. coli compound.

FIG. 9 is an HPLC profile showing the de novo production of styrene by yeast over-expressing PAL1 and FDC1. Peak 1 is styrene.

FIG. 10 an HPLC profile showing the de novo production of styrene by yeast expressing PAL2 and FDC1. Peak 1 is styrene.

FIG. 11 an HPLC profile showing the de novo production of styrene by yeast expressing PAL4 and FDC1. Peak 1 is styrene.

FIG. 12 is an HPLC profile of the products by wild-type E. coli after feeding on cinnamic acid.

FIG. 13 is an HPLC profile of the products by E. coli expressing PAD1 and feeding on cinnamic acid.

FIG. 14 is an HPLC profile of the products by E. coli expressing AnOHBA1 and feeding on cinnamic acid.

FIG. 15 shows the production of styrene in aqueous phase of culture media (in Panel A) after the consumption of cinnamic acid substrate (Panel B) by E. coli expressing either AnOHBA1 (designated by O) or PAD1 (designated by P) or wild-type E. coli (designated by Wt). Panel C shows the level of substrate remaining in the culture media at 2 and 5 hours after feeding E. coli with cinnamic acid.

FIG. 16 shows the production of styrene (in Panel A) by E. coli expressing either AnOHBA1 (designated by O) or FDC1 (designated by F) and feeding on cinnamic acid substrate (Panel B) for 2 or 4 hours.

FIG. 17 shows the production of styrene by feeding phenylalanine to yeast expressing PAL2 and FDC. The changes in pH, OD600 (in Panel A), phenylalanine substrate concentration, and the styrene production (in Panel B) are shown over the fermentation period.

FIG. 18 shows E. coli failing to express functional (soluble form of) FDC1 at 37° C. Three different lysis buffers (designated as A, B and C) were used to study the expression of FDC1 in E. coli strain BL-21 (DE3) after induction with IPTG at 37° C. and continued growth for 3 hours at the same temperature. As shown, the FDC1 protein was associated with the insoluble pellet fraction, indicating the formation of inactive, non-functional aggregates of the FDC1 protein, i.e., inclusion bodies.

FIG. 19 shows the expression of functional FDC1 in E. coli when the cells were induced with IPTG at 16° C., and continued to be cultured at the same temperature for 16 hours.

FIG. 20 shows the effect of metal ions on the activity of FDC1 enzyme. Specific activity is shown as nmol of styrene produced per mg of enzyme per minute.

FIG. 21 shows that metal chelation can reduce the enzyme activity of FDC1. Specific activity is shown as nmol of styrene produced per mg of enzyme per minute.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined, italicized and/or boldface headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

In describing and claiming the subject technology, the following terminology will be used in accordance with the definitions set out below. The following abbreviations and definitions will be used for the interpretation of the specification and the claims.

“Phenylalanine ammonia-lyase” is abbreviated as PAL.

“Ferulic acid decarboxylase” is abbreviated as FDC.

“Phenylacrylic acid decarboxylase” is abbreviated as PAD.

The term “PAL activity” refers to the ability of a protein to catalyze the conversion of phenylalanine to cinnamic acid. The terms “cinnamic acid” and “cinnamate” are used interchangeably in the specification.

The term “cinnamic acid decarboxylate activity” refers to the ability of a protein to catalyze the conversion of cinnamic acid to styrene.

The term “phenylalanine over-producing strain” refers to a microbial strain that produces endogenous levels of phenylalanine significantly higher than those seen in the wild-type of that strain. One specific example of an E. coli phenylalanine over-producer is the E. coli strain NST74 (U.S. Pat. No. 4,681,852, the entirety of which is hereby incorporated herein by reference). Others may include Corynebacterium glutamicum (Ikeda, M. and Katsumata, R. Metabolic engineering to produce tyrosine or phenylalanine in a tryptophan-producing Corynebacterium glutamicum strain, Appl. Environ. Microbiol. (1992), 58(3), pp. 781 785, the entirety of which is hereby incorporated herein by reference).

The term “fermentable carbon substrate” or “fermentable carbon source” refers to a carbon source capable of being metabolized by host organisms of the subject technology. Exemplary carbon sources include monosaccharides, oligosaccharides, polysaccharides, one-carbon substrates or mixtures thereof.

The term “complementary” is to be given its ordinary and customary meaning to a person of ordinary skill in the art, and is used without limitation to describe the relationship between nucleotide bases that are capable to hybridizing to one another. For example, with respect to DNA, adenosine is complementary to thymine and cytosine is complementary to guanine. Accordingly, the subjection technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art, and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art, and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

A fragment can also be a “functional fragment,” in which case the fragment retains some or all of the activity of the reference polypeptide as described herein. For example, in some embodiments, a functional fragment of a PAL and/or FDC polypeptide can retain some or all of the ability of the reference polypeptides to catalyze the phenylalanine to cinnamic acid conversion and/or the cinnamic acid to styrene conversion, respectively, such as what had been described in the present application for full-length PAL and/or FDC.

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide. For example, a variant of a PAL and/or FDC polypeptide includes an amino acid sequence that is different from the PAL and/or FDC reference polypeptides by one or more amino acids (e.g., by one or more amino acid substitutions, deletions, and/or additions) but can retain some or all of the activities of the reference polypeptides to catalyze the cinnamic acid and/or the styrene synthesis, respectively. A variant of a reference polypeptide also refers to a variant of a fragment of the reference polypeptide, for example, a fragment wherein one or more amino acid substitutions have been made relative to the reference polypeptide. A variant also includes a “functional fragment” of a reference polypeptide. The term “functional fragment is defined above.

The term functional variant further includes conservatively substituted variants. The term “conservatively substituted variant” refers to a peptide comprising an amino acid residue sequence that differs from a reference peptide by one or more conservative amino acid substitutions, and maintains some or all of the activity of the reference peptide as described herein. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide (as defined below) having at least about 80% amino acid sequence identity with the amino acid sequence of SEQ ID NO:2, SEQ ID NO:4, SEQ ID NO:6, SEQ ID NO:8, or SEQ ID NO: 20 over the entire length of the shorter of the two sequences. Such variant polypeptides include, for instance, polypeptides wherein one or more amino acid residues are added, or deleted, at the N- and/or C-terminus, as well as within one or more internal domains, of SEQ ID NO:2, SEQ ID NO:4, SEQ ID NO:6, SEQ ID NO:8, or SEQ ID NO: 20. Ordinarily, a variant polypeptide of the subject technology will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with the amino acid sequence of SEQ ID NO:2, SEQ ID NO:4, SEQ ID NO:6, SEQ ID NO:8, or SEQ ID NO: 20, over the entire length of the shorter of the two sequences.

“Percent (%) amino acid sequence identity” with respect to the variant polypeptide sequences of the subject technology is defined as the percentage of amino acid residues in a candidate sequence that are identical with the amino acid residues of SEQ ID NO:2, SEQ ID NO:4, SEQ ID NO:6, SEQ ID NO:8, or SEQ ID NO: 20, after aligning the sequences and introducing gaps, if necessary, to achieve the maximum percent sequence identity, and not considering any conservative substitutions as part of the sequence identity.

Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, the % amino acid sequence identity may be determined using the sequence comparison program NCBI-BLAST2. The NCBI-BLAST2 sequence comparison program may be downloaded from ncbi.nlm.nih.gov. NCBI BLAST2 uses several search parameters, wherein all of those search parameters are set to default values including, for example, unmask yes, strand=all, expected occurrences 10, minimum low complexity length=15/5, multi-pass e-value=0.01, constant for multi-pass=25, dropoff for final gapped alignment=25 and scoring matrix=BLOSUM62. In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The term “variant,” in connection with the polynucleotides of the subject technology, further refers to a nucleic acid sequence which encodes an active phenylalanine ammonia lyase (PAL) such as that with an amino acid sequence listed in SEQ ID NO:2, SEQ ID NO:4 or SEQ ID NO:6; an active ferulic acid decarboxylase (FDC) such as that with an amino acid sequence listed in SEQ ID NO: 8; or an active A. niger OHBA1 (AnOHBA1) polypeptide such as that with an amino acid sequence listed in SEQ ID NO:20. Alternatively, the term variant in this context refers to a nucleic acid sequence having at least about 80% nucleic acid sequence identity with either (a) SEQ ID NO:1, SEQ ID NO:3, SEQ ID NO:5, SEQ ID NO:7, or SEQ ID NO:19, over the entire length of the sequence, or (b) a nucleic acid sequence which encodes SEQ ID NO:2, SEQ ID NO:4, SEQ ID NO:6, SEQ ID NO:8, or SEQ ID NO: 20, over the entire length of the sequence.

“Percent (%) nucleic acid sequence identity” with respect to the variant polynucleotides of the subject technology is defined as the percentage of nucleotides in a candidate sequence that are identical with the nucleotides in either (a) SEQ ID NO:1, SEQ ID NO:3, SEQ ID NO:5, SEQ ID NO:7 or SEQ ID NO:19, or (b) a nucleic acid sequence which encodes SEQ ID NO:2, SEQ ID NO:4, SEQ ID NO:6, SEQ ID NO:8, or SEQ ID NO: 20; after aligning the sequences and introducing gaps, if necessary, to achieve the maximum percent sequence identity.

Alternatively, the term “variant,” in connection with the polynucleotides of the subject technology, refers to a nucleic acid sequence which can hybridize, under a stringent hybridization condition, to either of the two complementary strands of SEQ ID NO:1, SEQ ID NO:3, SEQ ID NO:5, SEQ ID NO:7, or SEQ ID NO:19 and encodes a polypeptide which has a phenylalanine ammonia lyase or a cinnamic acid decarboxylase activity.

As used herein, the term “stringent hybridization and wash conditions” describes conditions for hybridization and washing. Stringent conditions are known to those skilled in the art and can be found in available references (e.g., Current Protocols in Molecular Biology, John Wiley & Sons, N.Y., 1989, 6.3.1-6.3.6). Aqueous and non-aqueous methods are described in that reference and either can be used. An example of stringent hybridization conditions are hybridization in 6× sodium chloride/sodium citrate (SSC) at about 45° C., followed by one or more washes in 0.2×SSC, 0.1% (w/v) SDS at 50° C. Another example of stringent hybridization conditions are hybridization in 6×SSC at about 45° C., followed by one or more washes in 0.2×SSC, 0.1% (w/v) SDS at 55° C. A further example of stringent hybridization conditions are hybridization in 6×SSC at about 45° C., followed by one or more washes in 0.2×SSC, 0.1% (w/v) SDS at 60° C. Another example of stringent hybridization conditions are hybridization in 6×SSC at about 45° C., followed by one or more washes in 0.2×SSC, 0.1% (w/v) SDS at 65° C. Another example of stringent hybridization conditions are 0.5 molar sodium phosphate, 7% (w/v) SDS at 65° C., followed by one or more washes at 0.2×SSC, 1% (w/v) SDS at 65° C.

“Coding sequence” is to be given its ordinary and customary meaning to a person of ordinary skill in the art, and is used without limitation to refer to a DNA sequence that encodes for a specific amino acid sequence.

“Suitable regulatory sequences” is to be given its ordinary and customary meaning to a person of ordinary skill in the art, and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art, and is used without limitation to refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” is to be given its ordinary and customary meaning to a person of ordinary skill in the art, and is used without limitation to refer to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, is to be given its ordinary and customary meaning to a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“RNA transcript” is to be given its ordinary and customary meaning to a person of ordinary skill in the art, and is used without limitation to refer to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell.

“Antisense RNA” is to be given its ordinary and customary meaning to a person of ordinary skill in the art, and is used without limitation to refer to a RNA transcript that is complementary to all or part of a target primary transcript or mRNA and that blocks the expression of a target gene (U.S. Pat. No. 5,107,065). The complementary of an antisense RNA may be with any part of the specific gene transcript, i.e., at the 5′ non-coding sequence, 3′ non-coding sequence, introns, or the coding sequence.

“Transformation” is to be given its ordinary and customary meaning to a person of ordinary skill in the art, and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “recombinant” or “transformed” organisms.

The terms “transformed,” “transgenic,” and “recombinant,” when used herein in connection with host cells, are to be given their respective ordinary and customary meanings to a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “heterologous,” “recombinant,” and “exogenous,” when used herein in connection with polynucleotides, are to be given their ordinary and customary meanings to a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found. Similarly, when used in the context of a polypeptide or amino acid sequence, an exogenous polypeptide or amino acid sequence is a polypeptide or amino acid sequence that originates from a source foreign to the particular host cell or, if from the same source, is modified from its original form. Thus, exogenous DNA segments can be expressed to yield exogenous polypeptides.

The terms “plasmid”, “vector” and “cassette” are to be given their respective ordinary and customary meanings to a person of ordinary skill in the art, and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

Standard recombinant DNA and molecular cloning techniques used here are well known in the art and are described by Sambrook, J., Fritsch, E. F. and Maniatis, T. Molecular Cloning: A Laboratory Manual, 2.sup.nd ed.; Cold Spring Harbor Laboratory: Cold Spring Harbor, N.Y., 1989 (hereinafter “Maniatis”); and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by Greene Publishing and Wiley-Interscience, 1987; the entirety of each of which is hereby incorporated herein by reference.

Following long-standing patent law convention, the terms “a”, “an”, and “the” refer to “one or more” when used in this application, including the claims. Thus, for example, reference to “a cell” includes a plurality of such cells, and so forth.

In some embodiments, the subject technology relates to a method for production of styrene comprising (i) contacting a recombinant host cell with a fermentable substrate (such as a carbon source or a nitrogen source), said recombinant host cell comprising (a) at least one polynucleotide or gene encoding a polypeptide having phenylalanine ammonia-lyase activity, and (b) at least one polynucleotide or gene encoding a polypeptide having cinnamic acid decarboxylase activity; and (ii) growing said recombinant host cell for a time sufficient to produce styrene. In an aspect relating to this embodiment, the polynucleotide or gene encodes a polypeptide having phenylalanine ammonia-lyase activity, such as a phenylalanine ammonia-lyase (PAL) whose amino acid sequence is set forth in SEQ ID NO:2, SEQ ID NO:4, or SEQ ID NO:6, or a variant thereof. In an aspect relating to this embodiment, the polynucleotide or gene encodes a polypeptide having cinnamic acid decarboxylase activity, such as (a) ferulic acid decarboxylase (FDC) whose amino acid sequence is set forth in SEQ ID NO:8, or a variant thereof, or (b) AnOHBA1 whose amino acid sequence is set forth in SEQ ID NO:20, or a variant thereof. In an embodiment, the recombinant host cell of the subject technology does not comprise a polynucleotide or gene encoding a phenylacrylic acid decarboxylase (PAD1), whose amino acid sequence is set forth in SEQ ID NO:9.

In an embodiment, the recombinant host cell of the subject technology does not comprise a polynucleotide or gene encoding a functional phenylacrylic acid decarboxylase (PAD1) whose amino acid sequence is set forth in SEQ ID NO: 10, SEQ ID NO:16 or SEQ ID NO:18.

In some embodiments, the subject technology relates to a recombinant host cells comprising (a) at least one polynucleotide or gene encoding a polypeptide having phenylalanine ammonia-lyase activity, and (b) at least one polynucleotide or gene encoding a polypeptide having a cinnamic acid decarboxylase activity. In an aspect relating to this embodiment, the polynucleotide or gene encodes a polypeptide having phenylalanine ammonia-lyase activity, such as a PAL whose sequence is set forth in SEQ ID NO:2, SEQ ID NO:4, or SEQ ID NO:6, or a variant thereof. In an aspect relating to this embodiment, the polynucleotide or gene encodes a polypeptide having cinnamic acid decarboxylase activity, such as (a) ferulic acid decarboxylase (FDC) whose amino acid sequence is set forth in SEQ ID NO:8, or a variant thereof, or (b) AnOHBA1 whose amino acid sequence is set forth in SEQ ID NO:20, or a variant thereof. In an embodiment, the recombinant host cell of the subject technology does not comprise a polynucleotide or gene encoding a phenylacrylic acid decarboxylase (PAD), whose amino acid sequence is set forth in SEQ ID NO:10, SEQ ID NO:16 or SEQ ID NO:18.

In some embodiments, the subject technology relates to a microbial host which is recombinantly engineered to produce styrene. In an embodiment, the microbial host comprises at least one PAL polynucleotide or gene encoding a PAL polypeptide, and at least one of FDC polynucleotide or gene encoding an FDC polypeptide or AnOHBA1 polynucleotide encoding an ANOHBA1 polypeptide. In a related embodiment, the microbial host does not comprise a polynucleotide or gene encoding a PAD polypeptide.

Therefore, the subject technology provides an inexpensive biological route to production of styrene which is useful in a variety of commercial materials including polystyrene, ABS, styrene-butadiene (SBR) rubber, styrene-butadiene latex, SIS (styrene-isoprene-styrene), S-EB-S (styrene-ethylene/butylene-styrene), styrene-divinylbenzene (S-DVB), styrene-acrylonitrile resin (SAN) and unsaturated polyesters.

Since styrene is an essential compound used in many products, alternative production methods, especially ones that do not require fossil fuels as feed stock, is urgently needed. Biological synthesis of styrene using renewable material will be very important to sustained economic development.

However, the metabolic pathway that leads to styrene biosynthesis in plants has not been delineated. At the time of preparation of this application, a search of PubMed with keyword “styrene biosynthesis” or “styrene pathway” yielded zero publications. Exactly how styrene is synthesized in bio-organisms is largely unknown.

In 2010, PCT Publication No. WO2010/053950 (PCT Application No. PCT/US2009/063219) reported the bioconversion of feed substrate to styrene and other compounds by a number of micro-organisms, including filamentous fungi. However, only the native production by several fungi was scaled up in this patent, no molecular mechanisms of the biosynthesis were investigated, and the yield of this native production was shown to be extremely low.

On the other hand, biosynthesis of p-hydroxystyrene has been reported previously. In 2007, Qi et al. reported that functional expression of both prokaryotic and eukaryotic genes in E. coli can be used to produce p-hydroxystyrene from glucose (Metabolic Engineering 9 (2007): 268-276). In that report, Qi et al. used tyrosine/phenylalanine ammonia-lyase (PAL/TAL) enzyme (from yeast Rhodotorula glutinis) to yield p-hydroxycinnamic acid (pHCA). Subsequently, Qi et al. used pHCA decarboxylase (PDC) from a Bacillus subtilis to produce p-hydroxystyrene from pHCA. In the Qi et al. study, cinnamic acid was accumulated in the culture media due to the activity of PAL. However, the conversion of cinnamic acid to styrene was not reported, suggesting that the PDC enzyme did not have a cinnamic acid decarboxylase activity to convert the cinnamic acid to styrene.

Similarly, in 2009, Verhoef et al. reported the p-hydroxystyrene from glucose by Pseudommonas putida (Applied and Environmental Microbiology 75 (2009): 931-936). In that case, Verhoef et al. used a PAL/TAL gene (from yeast Rhodotorula glutinis) and a p-coumaric acid decarboxylase gene (from Lactobaillus plantarum) to biosynthesize p-hydroxystyrene. Verhoef et al. did not suggest or report production of styrene in their study.

In 2010, a paper by Landete et al. was published in the Journal of Industrial Microbiology and Biotechnology (vol. 37 (2010): 617-624) which reported the cloning, expression, and characterization of phenolic acid decarboxylase (PAD) from Lactobacillus brevis. Similar to previous publications, the report focused on PAD's activity in catalyzing the decarboxylation of phenolic compound. Once again, the enzyme was shown to convert p-coumaric acid, caffeic acid, and ferulic acid into their corresponding vinyl phenols. No mention of styrene biosynthesis was made in that paper.

To date, Applicant is aware of one EPA grant proposal described on US Environmental Protection Agency's website titled “Final Report: Engineering the biosynthesis of styrene in yeast,” with project period Aug. 31, 2007 to Jul. 31, 2008; in which the authors from Carnegie Mellon University suggested using a combination of PAL and PAD (phenolic acid decarboxylase) gene to produce styrene. However, as shown in the Examples below, it was demonstrated that combination of PAL and PAD will not produce styrene.

Therefore, the subject technology is, in part, based on the surprising finding that when a plant PAL polynucleotide and a yeast FDC polynucleotide are co-expressed in a host cell, such as yeast or bacteria, styrene can be produced. It was determined that phenylalanine ammonium lyase (PAL) catalyzes the synthesis of cinnamic acid from phenylalanine, and ferulic acid decarboxylase (FDC) catalyzes the conversion of cinnamic acid to styrene.

A. Polynucleotide of the Subject Technology

The amino acid and DNA sequences of the enzymes used in the subject technology are known in the art. U.S. Pat. Nos. 5,955,137 and 6,468,566 (the entirety of both of which are hereby incorporated herein by reference in their entirety) describe isolation and sequencing of ferulic acid decarboylase (FDC) which, as described by these patents, is used for 4-vinylguaiacol (4-VG), vanillin, or vanillic acid production in liquor industry, not for production of styrene.

Polynucleotides or genes encoding PAL polypeptide are also known in the art and several have been sequenced from both plant and microbial sources. For example, a PAL gene obtained from R. toruloides in EP 321488; from Eucalyptus grandis and Pinus radiate in WO 9811205; from Petunia in WO 9732023; from Pisum sativum in JP 05153978; and from potato and rice in WO 9307279 (the entire contents of each of the foregoing patent documents are hereby incorporated herein by reference in their entirety). The cDNA sequence of PAL is publically available (see for example GENBANK® Accession Nos. AJ010143 and X75967). Where expression of a wild type PAL polynucleotide in a recombinant host is desired the wild type gene may be obtained from any source including but not limited to, yeasts such as Rhodotorula sp., Rhodosporidium sp. and Sporobolomyces sp.; bacterial organisms such as Streptomyces; and plants such as pea, potato, rice, eucalyptus, pine, corn, petunia, Arabidopsis, tobacco, and parsley. For example, full-length cDNAs of Arabidopsis PAL1 (At2g37040), PAL2 (At3g53260) and PAL4 (At3g10340) used in the Examples below were purchased from Arabidopsis Biological Resource Center (ABRC) at the Ohio State University under the stock numbers G10120 (in pENTR223.1 vector), G12256 (in pENTR223.1 vector), and U24927 (in pENTR/D-TOPO vector), respectively.

It will be appreciated that the subject technology is not limited to the polynucleotides mentioned above, but will encompass any variant polynucleotide of PAL, FDC, or OHBA1 that may be obtained by routine molecular biology techniques.

Accordingly, in some embodiments, the polynucleotide of the subject technology comprises a variant polynucleotide of a PAL-coding sequence as described herein. In an embodiment, the polynucleotide of the subject technology comprises a variant of a polynucleotide sequence as set forth in SEQ ID NO:1, SEQ ID NO:3, or SEQ ID NO:5.

In some embodiments, the polynucleotide of the subject technology comprises a variant polynucleotide of FDC. In an embodiment, the polynucleotide of the subject technology comprises a variant polynucleotide sequence as set forth in SEQ ID NO:7, or SEQ ID NO:19.

In an embodiment, the variant polynucleotide of the subject technology comprises a nucleic acid sequence which encodes an active phenylalanine ammonia lyase (PAL) such as that shown in SEQ ID NO:2, SEQ ID NO:4 or SEQ ID NO:6 (that can convert phenylalanine to cinnamic acid); an active ferulic acid decarboxylase (FDC) such as that shown in SEQ ID NO: 8, or an active A. niger OHBA1 (AnOHBA1) polypeptide such as that shown in SEQ ID NO:20 (which can convert cinnamic acid to styrene). In an embodiment, a variant polynucleotide of the subject technology comprises a nucleic acid sequence having at least about 80% nucleic acid sequence identity with either (a) SEQ ID NO:1, SEQ ID NO:3, SEQ ID NO:5, SEQ ID NO:7, or SEQ ID NO:19 (over the entire length of the shorter of the two sequences) or (b) a nucleic acid sequence which encodes SEQ ID NO:2, SEQ ID NO:4, SEQ ID NO:6, SEQ ID NO:8, or SEQ ID NO: 20 (over the entire length of the shorter of the two sequences).

In an embodiment, the variant polynucleotide of the subject technology comprises at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity, with either (a) SEQ ID NO:1, SEQ ID NO:3, SEQ ID NO:5, SEQ ID NO:7 or SEQ ID NO:19 (over the entire length of the sequence), or (b) a nucleic acid sequence which encodes SEQ ID NO:2, SEQ ID NO:4, SEQ ID NO:6, SEQ ID NO:8, or SEQ ID NO: 20 (over the entire length of the sequence).

Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, the % nucleic acid sequence identity may be determined using the sequence comparison program NCBI-BLAST2 (Altschul et al., Nucleic Acids Res. 25:3389-3402 (1997)). The NCBI-BLAST2 sequence comparison program may be downloaded from ncbi.nlm.nih.gov. NCBI BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62. In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In some embodiments, the variant polynucleotide of the subject technology comprises a nucleic acid sequence that encode an active PAL or FDC or OHBA1 polypeptide and which is capable of hybridizing, preferably under stringent hybridization and wash conditions, to a nucleotide sequence encoding a full-length polypeptide shown in SEQ ID NO:2, SEQ ID NO:4, SEQ ID NO:6, SEQ ID NO:8, or SEQ ID NO: 20.

Methods of obtaining homologous and/or variant polynucleotides using sequence-dependant protocols are well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction (PCR), ligase chain reaction (LCR)).

For example, polynucleotides or genes encoding variants of anyone of the polypeptides having the above mentioned activities could be isolated directly by using all or a portion of the known sequences as DNA hybridization probes to screen libraries from any desired plant, fungi, yeast, or bacteria using methodology well known to those skilled in the art. Specific oligonucleotide probes based upon the literature nucleic acid sequences can be designed and synthesized by methods known in the art (Maniatis, infra.). Moreover, the entire sequences can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of or full-length of the instant sequences. The resulting amplification products can be labeled directly during amplification reactions or labeled after amplification reactions, and used as probes to isolate full-length cDNA or genomic fragments under conditions of appropriate stringency.

In addition, two short segments of the literature sequences (e.g., SEQ ID NO:1, SEQ ID NO:3, SEQ ID NO:5, SEQ ID NO:7 or SEQ ID NO:19) may be used as primers in polymerase chain reaction protocols to amplify longer nucleic acid fragments encoding variant polynucleotides from DNA or RNA. The polymerase chain reaction may also be performed on a library of cloned nucleic acid fragments wherein the sequence of one primer is derived from the literature sequences, and the sequence of the other primer takes advantage of the presence of the polyadenylic acid tracts to the 3′ end of the mRNA precursor encoding bacterial genes. Alternatively, the second primer sequence may be based upon sequences derived from the cloning vector. For example, the skilled artisan can follow the RACE protocol (Frohman et al., PNAS USA 85:8998 (1988), the entirety of which is hereby incorporated herein by reference in its entirety) to generate cDNAs by using PCR to amplify copies of the region between a single point in the transcript and the 3′ or 5′ end. Primers oriented in the 3′ and 5′ directions can be designed from the literature sequences. Using commercially available 3′ RACE or 5′ RACE systems, specific 3′ or 5′ cDNA fragments can be isolated (Ohara et al., Proc. Natl. Acad. Sci. USA 86:5673 (1989); Loh et al., Science 243:217 (1989), the entirety of each of which is hereby incorporated herein by reference in its entirety).

B. Polypeptides of the Subject Technology

In some embodiments, the subject technology relates to a polypeptide of SEQ ID NO:2, SEQ ID NO:4, SEQ ID NO:6, SEQ ID NO:8, or SEQ ID NO: 20. In an embodiment, the subject technology relates to a polypeptide having at least about 70%; or at least about 80%, or at least about 85%, or at least about 90%, or at least about 95%, or at least about 99% sequence identity to a polypeptide of the subject technology (e.g., PAL1 which is set forth in SEQ ID NO:2; PAL2 which is set forth in SEQ ID NO:4; PAL4 which is set forth in SEQ ID NO:6; FDC which is set forth in SEQ ID NO:8; or AnOHBA1 which is set forth in SEQ ID NO:20).

In some embodiments, the polypeptide of the subject technology comprises a variant polypeptide of PAL. In an embodiment, the polypeptide of the subject technology comprises a variant polypeptide of PAL1 which is set forth in SEQ ID NO:2, a variant polypeptide of PAL2 which is set forth in SEQ ID NO:4, or a variant polypeptide of PAL4 which is set forth in SEQ ID NO:6.

In some embodiments, the polypeptide of the subject technology comprises a variant polypeptide of FDC. In an embodiment, the polypeptide of the subject technology comprises a variant polypeptide of FDC which is set forth in SEQ ID NO:8, or a variant polypeptide of AnOHBA1 which is set forth in SEQ ID NO:20.

In an embodiment, the variant polypeptide of the subject technology comprises an active phenylalanine ammonia lyase (PAL) polypeptide such as that shown in SEQ ID NO:2, SEQ ID NO:4 or SEQ ID NO:6 (that can convert phenylalanine to cinnamic acid); an active ferulic acid decarboxylase (FDC) such as that shown in SEQ ID NO: 8 or an active A. niger OHBA1 (AnOHBA1) polypeptide such as that shown in SEQ ID NO:20 (which can convert cinnamic acid to styrene).

In an embodiment, the variant polypeptide of the subject technology comprises a polypeptide having at least about 80% amino acid sequence identity with the amino acid sequence of SEQ ID NO:2, SEQ ID NO:4, SEQ ID NO:6, SEQ ID NO:8, or SEQ ID NO: 20 over the entire length of the sequence.

In an embodiment, the variant polypeptide of the subject technology comprises a polypeptide having at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with the amino acid sequence of SEQ ID NO:2, SEQ ID NO:4, SEQ ID NO:6, SEQ ID NO:8, or SEQ ID NO: 20, over the entire length of the sequence.

Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, the % amino acid sequence identity may be determined using the sequence comparison program NCBI-BLAST2 (Altschul et al., Nucleic Acids Res. 25:3389-3402 (1997)). The NCBI-BLAST2 sequence comparison program may be downloaded from ncbi.nlm.nih.gov. NCBI BLAST2 uses several search parameters, wherein all of those search parameters are set to default values including, for example, unmask yes, strand=all, expected occurrences 10, minimum low complexity length=15/5, multi-pass e-value=0.01, constant for multi-pass=25, dropoff for final gapped alignment=25 and scoring matrix=BLOSUM62. In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

In an embodiment, the PAL1, PAL2, PAL4, FDC or AnOHBA1 polypeptides of the subject technology each have an amino acid sequence sufficiently homologous to the amino acid sequence of SEQ ID NO:2, SEQ ID NO:4, SEQ ID NO:6, SEQ ID NO:8, SEQ ID NO:20, respectively. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains and/or a common functional activity. For example, amino acid or nucleotide sequences which share common structural domains and/or common functional activity have at least about 40% homology, or 50% homology, or 60%-70% homology, or 70%-80% homology, or 80%-95% homology across the entire amino acid or nucleotide sequences of the proteins of the subject technology. Furthermore, amino acid or nucleotide sequences which share at least about 60%, or about 70%, or about 75%, or about 80%, or about 85%, or about 90%, or about 95% homology and share a common functional activity are defined herein as sufficiently homologous.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In one embodiment, an alignment is a global alignment, e.g., an overall sequence alignment. In another embodiment, an alignment is a local alignment. In a preferred embodiment, the length of a sequence aligned for comparison purposes is at least 30%, preferably at least 40%, more preferably at least 50%, even more preferably at least 60%, and even more preferably at least 70%, 80%, or 90% of the length of the reference sequence to which it is aligned (e.g., when aligning a second sequence to the FDC amino acid sequence of SEQ ID NO:8, at least 151, preferably at least 201, more preferably at least 252, even more preferably at least 302, and even more preferably at least 353, 403 or 453 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each cap, which need to be introduced for optimal alignment of the two sequences.

C. Fusion Proteins of the Subject Technology

In some embodiments, the subject technology comprises PAL, FDC, and/or AnOHBA1 chimeric or fusion proteins. As used herein, a “chimeric protein” or “fusion protein” comprises a PAL, FDC, or AnOHBA1 polypeptide operatively linked to a non-PAL polypeptide, non-FDC polypeptide or non-AnOHBA1 polypeptide, respectively. For example, A “PAL polypeptide” or “FDC polypeptide” refers to a polypeptide having an amino acid sequence corresponding to a PAL or FDC proteins of the subject technology, respectively, whereas a “non-PAL polypeptide” or “non-FDC polypeptide” refers to a polypeptide having an amino acid sequence corresponding to a protein which is not substantially homoloeous or identical to the PAL or FDC protein, e.g., a protein which is different from the PAL or FDC protein and which is derived from the same or a different organism. Within a PAL or FDC fusion protein, the PAL or FDC polypeptide can correspond to all or a portion of a PAL or FDC protein. In an embodiment, a PAL or FDC fusion protein comprises at least one biologically active portion of a PAL protein. In an embodiment, a PAL or FDC fusion protein comprises at least two biologically active portions of a PAL or FDC protein. In an embodiment, a PAL or FDC fusion protein comprises at least three biologically active portions of a PAL or FDC protein. Within the fusion protein, the term “operatively linked” is intended to indicate that the PAL or FDC polypeptide and the non-PAL or non-FDC polypeptide are fused in-frame to each other. The non-PAL or non-FDC polypeptide can be fused to the N-terminus or C-terminus of the PAL or FDC polypeptide.

For example, in an embodiment, the fusion protein is a GST-FDC fusion protein in which the FDC sequences are fused to the C-terminus of the GST sequences. Such fusion proteins can facilitate the purification of recombinant FDC.

In some embodiments, the subject technology relates to the PAL or FDC polypeptides having been mutagenized or engineered for obtaining improved enzymes. A variety of approaches may be used for the mutagenesis of the PAL or FDC enzyme. Two suitable approaches used herein include error-prone PCR (Leung et al., Techniques, 1:11 15 (1989) and Zhou et al., Nucleic Acids Res. 19:6052 6052 (1991) and Spee et al., Nucleic Acids Res. 21:777 778 (1993); the entirety of each of which is hereby incorporated herein by reference in its entirety) and in vivo mutagenesis.

The principal advantage of error-prone PCR is that all mutations introduced by this method will be within the PAL polynucleotide, and any change may be easily controlled by changing the PCR conditions. Alternatively in vivo mutagenesis, may be employed using commercially available materials such as E. coli XL1-Red strain, and the Epicurian coli XL1-Red mutator strain from Stratagene (Stratagene, La Jolla, Calif., Greener and Callahan, Strategies 7:32 34 (1994)). This strain is deficient in three of the primary DNA repair pathways (mutS, mutD and mutT), resulting in a mutation rate 5000-fold higher than that of wild type. In vivo mutagenesis does not depend on ligation efficiency (as with error-prone PCR), however a mutation may occur at any region of the vector and the mutation rates are generally much lower.

D. Expression Vectors of the Subject Technology

In some embodiments, the subject technology relates to an expression vector comprising at least one polynucleotide of the subject technology and wherein the expression vector, upon transfection into a host cell, is capable of expressing at least one polypeptide of the subject technology. In an embodiment, the expression vector of the subject technology comprises at least one of PAL1, PAL2, PAL4, FDC1, AnOHBA1 polynucleotide or a variant thereof. In an embodiment, the expression vector of the subject technology comprises at least SEQ ID NO:1 or a variant thereof. In an embodiment, the expression vector of the subject technology comprises at least SEQ ID NO:3 or a variant thereof. In an embodiment, the expression vector of the subject technology comprises at least SEQ ID NO:5 or a variant thereof. In an embodiment, the expression vector of the subject technology comprises at least SEQ ID NO:7 or a variant thereof. In an embodiment, the expression vector of the subject technology comprises at least SEQ ID NO:19 or a variant thereof. In an embodiment, the expression vector of the subject technology comprises at least one of SEQ ID NO:1, NO:3, SEQ ID NO:5, SEQ ID NO:7, SEQ ID NO:19, or a variant thereof.

The design of expression vector depends on such factors as the choice of the host cell to be transformed, the level of expression of protein desired, and the like. The expression vectors of the subject technology can be introduced into host cells to thereby produce proteins or polypeptides, including fusion proteins or polypeptides, encoded by the polynucleotides of the present disclosure (e.g., the PAL1 polypeptide encoded by SEQ ID NO:1; the PAL2 polypeptide encoded by SEQ ID NO:3; the PAL4 polypeptide encoded by SEQ ID NO:5; the FDC1 polypeptide encoded by SEQ ID NO:7, or the AnOHBA1 polypeptide encoded by SEQ ID NO:19).

Expression of proteins in prokaryotes is most often carried out in a bacterial host cell with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the subject technology.

In an embodiment, the expression vector of the subject technology comprises those genetic elements which are necessary for expression of the PAL, FDC, AnOHBA1 proteins in the bacterial cell. The elements required for transcription and translation in the bacterial cell include a promoter, a coding region for the protein complex, and a transcriptional terminator.

In an embodiment, the expression vectors of the subject technology comprise bacterial expression vectors, for example recombinant bacteriophage DNA, plasmid DNA or cosmid DNA, yeast expression vectors e.g. recombinant yeast expression vectors, vectors for expression in insect cells, e.g., recombinant virus expression vectors, for example baculovirus, or vectors for expression in plant cells, e.g. recombinant virus expression vectors such as cauliflower mosaic virus, CaMV, tobacco mosaic virus, TMV, or recombinant plasmid expression vectors such as Ti plasmids.

In an embodiment, the vector of the subject technology comprises a bacterial expression vector. In a related embodiment, the expression vector comprises a high-copy-number expression vector; alternatively, the expression vector comprise a low-copy-number expression vector, for example, a Mini-F plasmid.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR).

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector comprising ligation-independent cloning (LIC) sites can be employed. The required PCR amplified polynucleotide can then be cloned into the LIC vector without restriction digest or ligation (Aslanidis and de Jong, Nucl. Acid. Res. 18, 6069-6074, (1990), Haun, et al, Biotechniques 13, 515-518 (1992); the entirety of each of which is hereby incorporated herein by reference in its entirety).

In order to isolate and/or modify the polynucleotide of interest for insertion into the chosen plasmid, it is preferable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment a polynucleotide for incorporation into an expression vector of the subject technology, is prepared by the use of the polymerase chain reaction, using appropriate oligonucleotide primers. The coding region is amplified, whilst the primers themselves become incorporated into the amplified sequence product. In an embodiment, the amplification primers contain restriction endonuclease recognition sites which allow the amplified sequence product to be cloned into an appropriate vector.

In an embodiment, the polynucleotide of SEQ ID NO:1 or a variant thereof is obtained by PCR and introduced into an expression vector using restriction endonuclease digestion and ligation, a technique which is well known in the art. Alternatively or in addition, the polynucleotide of SEQ ID NO:3 or a variant thereof is obtained by PCR and introduced into an expression vector using restriction endonuclease digestion and ligation, a technique which is well known in the art. Alternatively or in addition, the polynucleotide of SEQ ID NO:5 or a variant thereof is obtained by PCR and introduced into an expression vector using restriction endonuclease digestion and ligation, a technique which is well known in the art. Alternatively or in addition, the polynucleotide of SEQ ID NO:7 or a variant thereof is obtained by PCR and introduced into an expression vector using restriction endonuclease digestion and ligation, a technique which is well known in the art. Alternatively or in addition, the polynucleotide of SEQ ID NO:19 or a variant thereof is obtained by PCR and introduced into an expression vector using restriction endonuclease digestion and ligation, a technique which is well known in the art.

In an embodiment, the polynucleotide of at least one of SEQ ID NO:1; SEQ ID NO:3; SEQ ID NO:5; SEQ ID NO:7; SEQ ID NO:19 or a variant thereof is introduced into an expression vector by yeast homologous recombination (Raymon et al., Biotechniques. 26(1): 134-8, 140-1, 1999; the entirety of which is hereby incorporated herein it its entirety). Thus, the expression vectors of the subject technology comprises a single copy of the polynucleotide described previously, or multiple copies of the polynucleotides described previously.

In an embodiment, the expression vector of the subject technology comprises at least one of SEQ ID NO:1; SEQ ID NO:3; SEQ ID NO:5; SEQ ID NO:7; SEQ ID NO:19 or a variant thereof encoding at least one polypeptide of SEQ ID NO:2; SEQ ID NO:4; SEQ ID NO:6; SEQ ID NO:8; SEQ ID NO:20; or a variant thereof.

E. Microbial Hosts of the Subject Technology

The production organisms of the subject technology will include any organism capable of expressing the polynucleotide (e.g., PAL and FDC or PAL and/or AnOHBA1) of the subject technology for styrene production. Typically the production organism will be restricted to microorganisms and plants. In an embodiment of the subject technology, the host cells of the subject technology do not contain a polynucleotide such as that shown in SEQ ID NO:9, SEQ ID NO:15 or SEQ ID NO:17, that encodes a phenylacrylic acid decarboxylase (PAD) polypeptide.

Microorganisms useful in the subject technology for the production of styrene may include, but are not limited to bacteria, such as the enteric bacteria (Escherichia, and Salmonella for example) as well as Bacillus, Acinetobacter, Actinomycetes such as Streptomyces, Corynebacterium, Methanotrophs such as Methylosinus, Methylomonas, Rhodococcus and Pseudomona; Cyanobacteria, such as Rhodobacterand Synechocystis; yeasts, such as Saccharomyces, Zygosaccharomyces, Kluyveromyces, Candida, Hansenula, Debaryomyces, Mucor, Pichia and Torulopsis; and filamentous fungi such as Aspergillus and Arthrobotrys, and algae, and Escherichia, Klebsiella, Pantoea, Salmonella Corynebacterium, Clostridium, and Clostridium acetobutylicum, for example. The PAL and/or FDC polynucleotide of the subject technology may be incorporated into these and other microbial hosts for the host cells to prepare large, commercially useful amounts of styrene or other compounds downstream from styrene (compounds which will result from further processing of styrene in these microorganisms via enzymatic or biological pathways).

Although any of the above mentioned microorganisms would be useful in the production of styrene, preferred are mutant strains of bacteria that over-produce phenylalanine. The subject technology provides methods for the production of styrene using different combinations of genetic elements. In one case, a recombinant host may be constructed such that it expresses at least one polynucleotide encoding a polypeptide having phenylalanine ammonia lyase activity (i.e., a PAL polynucleotide) and at least one polynucleotide encoding a polypeptide having cinnamic acid decarboxylase activity (i.e., an FDC polynucleotide). In this situation it is preferred if the recombinant host over-express phenylalanine. Phenylalanine overproducing strains are known and include but are not limited to E. coli, Microbacterium Corynebacteria, Arthrobacter, Pseudomonas and Brevibacteria. Particularly useful phenylalanine overproducing strains include, but are not limited to Microbacterium ammoniaphilum ATCC 10155, Corynebactrium lillium NRRL-B-2243, Brevibacterium divaricatum NRRL-B-2311, E. coli NST74 and Arthrobacter citreus ATCC 11624. Other suitable phenylalanine overproducing strains are known and a review may be found in Metabolic Engineering For Microbial Production Of Aromatic Amino Acids And Derived Compounds, J. Bongaertes et al, Metabolic Engineering vol. 3, 289 300, 2001; the entirety of which is hereby incorporated herein by reference.

F. Plant Hosts of the Subject Technology:

According to the subject technology, in an embodiment, the nucleic acids of the subject technology may be used to create transgenic plants having the ability to express the necessary polypeptides for the production of styrene. Preferred plant hosts will be any variety that will support a high production level of the enzymes or polypeptides of the subject technology. Suitable green plants will include but are not limited to soybean, rapeseed (Brassica napus, B. campestris), sunflower (Helianthus annus), cotton (Gossypium hirsutum), corn, tobacco (Nicotiana tabacum), alfalfa (Medicago sativa), wheat (Triticum sp), barley (Hordeum vulgare), oats (Avena sativa), sorghum (Sorghum bicolor), rice (Oryza sativa), Arabidopsis, cruciferous vegetables (broccoli, cauliflower, cabbage, parsnips, etc.), melons, carrots, celery, parsley, tomatoes, potatoes, strawberries, peanuts, grapes, grass seed crops, sugar beets, sugar cane, beans, peas, rye, flax, hardwood trees, softwood trees, and forage grasses. Algal species include but not limited to commercially significant hosts such as Spirulina, Haemotacoccus, and Dunaliella. Suitable plants for the method of the subject technology also include biofuel, biomass, and bioenergy crop plants. Exemplary plants include Arabidopsis thaliana, rice (Oryza sativa), switchgrass (Panicum vigratum), Brachypodium spp, Brassica spp., and Crambe abyssinica.

In some embodiments, the subject technology comprises transgenic host cells or host that have been transformed with one or more of the vectors disclosed herein. As used herein, the term “plant cell” is understood to mean any cell derived from a monocotyledonous or a dicotyledonous plant and capable of constituting undifferentiated tissues such as calli, differentiated tissues such as embryos, portions of monocotyledonous plants, monocotyledonous plants or seed. The term “plant” is understood to mean any differentiated multi-cellular organism capable of photosynthesis, including monocotyledons and dicotyledons. In some embodiments, the plant cell can be an Arabidopsis plant cell, a tobacco plant cell, a soybean plant cell, a petunia plant cell, or a cell from another oilseed crop including, but not limited to, a canola plant cell, a rapeseed plant cell, a palm plant cell, a sunflower plant cell, a cotton plant cell, a corn plant cell, a peanut plant cell, a flax plant cell, and a sesame plant cell.

Alternatively, the hosts cells used in the subject technology may be those suitable for biofuel production including, but not limited to, single cell organisms, microorganisms, multicell organisms, plants, fungi, bacteria, algae, cultivated crops, non-cultivated crops, and/or the like.

G. Host Cell Transformation

The expression vectors of the subject technology can be introduced into plant or microbial host cells by conventional transformation or transfection techniques.

Transformation of appropriate cells with an expression vector of the subject technology is accomplished by methods known in the art and typically depends on both the type of vector and cell. Said techniques include, but are not limited to calcium phosphate or calcium chloride co-precipitation, DEAE-dextran-mediated transfection, lipofection, chemoporation or electroporation.

Successfully transformed cells, that is, those cells containing the expression vector of the subject technology, can be identified by techniques well known in the art. For example, cells transfected with an expression vector of the subject technology can be cultured to produce a polypeptide of the subject technology. Cells can be examined for the presence of the expression vector DNA by techniques well known in the art.

The host cell of the subject technology can contain a single copy of the expression vector described previously, or alternatively, multiple copies of the expression vector.

In some embodiment, the transformed cell is an animal cell, an insect cell, a plant cell, an algal cell, a fungal cell, or a yeast cell. In some embodiments, the cell is a plant cell selected from the group consisting of: canola plant cell, a rapeseed plant cell, a palm plant cell, a sunflower plant cell, a cotton plant cell, a corn plant cell, a peanut plant cell, a flax plant cell, a sesame plant cell, a soybean plant cell, and a petunia plant cell.

Microbial host cell's expression systems and expression vectors containing regulatory sequences that direct high level expression of foreign proteins are well known to those skilled in the art. Any of these could be used to construct vectors for expression of the polynucleotides of the subjection technology in a microbial host cell for the production of styrene. These vectors could then be introduced into appropriate microorganisms via transformation to allow for high level expression of the enzymes of the subject technology, e.g., PAL and/or FDC.

Vectors or cassettes useful for the transformation of suitable microbial host cells are well known in the art. Typically the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a styrene production host.

Initiation control regions or promoters, which are useful to drive expression of the relevant polynucleotide in the desired microbial host cell are numerous and familiar to those skilled in the art. Virtually any promoter capable of driving these genes is suitable for the subject technology including but not limited to CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, trp, IP_{L}, IP_{R}, T7, tac, and trc (useful for expression in Escherichia coli).

Termination control regions may also be derived from various genes native to the microbial hosts. Optionally, a termination site may be unnecessary, however, it is preferred if included.

In plant cells, overproduction of styrene may be accomplished by first constructing the expression vectors (including the polynucleotides) of the subject technology in which the coding region are operably linked to promoters capable of directing expression of a polynucleotide of the subject technology in the desired tissues at the desired stage of development. For reasons of convenience, the polynucleotides to be expressed may comprise promoter sequences and translation leader sequences derived from the same polynucleotide. 3′ Non-coding sequences encoding transcription termination signals should also be present. The polynucleotides of the subject technology may also comprise one or more introns in order to facilitate polynucleotide expression.

For plant host cells, any combination of any promoter and any terminator capable of inducing expression of a coding region may be used in the vector sequences of the subject technology. Some suitable examples of promoters and terminators include those from nopaline synthase (nos), octopine synthase (ocs) and cauliflower mosaic virus (CaMV) genes. One type of efficient plant promoter that may be used is a high level plant promoter. Such promoters, in operable linkage with a polynucleotide of the subject technology should be capable of promoting the expression of the polynucleotide. High level plant promoters that may be used in the subject technology include the promoter of the small subunit (ss) of the ribulose-1,5-bisphosphate carboxylase from example from soybean (Berry-Lowe et al., J. Molecular and App. Gen., 1:483 498 (1982), the entirety of which is hereby incorporated herein by its entirety), and the promoter of the chlorophyll a/b binding protein. These two promoters are known to be light-induced in plant cells (see, for example, Genetic Engineering of Plants, an Agricultural Perspective, A. Cashmore, Plenum, N.Y. (1983), pages 29 38; Coruzzi, G. et al., The Journal of Biological Chemistry, 258:1399 (1983), and Dunsmuir, P. et al., Journal of Molecular and Applied Genetics, 2:285 (1983), the entirety of each of which is hereby incorporated herein by reference).

The choice of plasmid vector depends upon the method that will be used to transform host plants. The skilled artisan is well aware of the genetic elements that must be present on the plasmid vector in order to successfully transform, select and propagate host cells containing the chimeric polynucleotide. The skilled artisan will also recognize that different independent transformation events will result in different levels and patterns of expression (Jones et al., EMBO J. 4:2411 2418 (1985); De Almeida et al., Mol. Gen. Genetics 218:78 86 (1989), the entirety of each of which is hereby incorporated herein by reference), and thus that multiple events must be screened in order to obtain lines displaying the desired expression level and pattern. Such screening may be accomplished by Southern analysis of DNA blots. Northern analysis of mRNA expression, Western analysis of protein expression, or phenotypic analysis.

Introduction of a polynucleotide of the subject technology into a plant cell can be performed by a variety of methods known to those of ordinary skill in the art including, but not limited to, insertion of a nucleic acid sequence of interest into an Agrobacterium rhizogenes Ri or Agrobacterium tumefaciens Ti plasmid, microinjection, electroporation, or direct precipitation. By way of providing an example, in some embodiments, transient expression of a polynucleotide of interest can be performed by agro-infiltration methods. In this regard, a suspension of Agrobacterium tumefaciens containing a polynucleotide of interest can be grown in culture and then injected into a plant by placing the tip of a syringe against the underside of a leaf while gentle counter-pressure is applied to the other side of the leaf. The Agrobacterium solution is then injected into the airspaces inside the leaf through stomata. Once inside the leaf, the Agrobacterium transforms the gene of interest to a portion of the plant cells where the gene is then transiently expressed.

As another example, transformation of a plasmid or polynucleotide of interest into a plant cell can be performed by particle gun bombardment techniques. In this regard, a suspension of plant embryos can be grown in liquid culture and then bombarded with plasmids or polynucleotides that are attached to gold particles, wherein the gold particles bound to the plasmid or nucleic acid of interest can be propelled through the membranes of the plant tissues, such as embryonic tissue. Following bombardment, the transformed embryos can then be selected using an appropriate antibiotic to generate new, clonally propagated, transformed embryogenic suspension cultures.

For additional guidance regarding methods of transforming and producing transgenic plant cells, see U.S. Pat. Nos. 4,459,355; 4,536,475; 5,464,763; 5,177,010; 5,187,073; 4,945,050; 5,036,006; 5,100,792; 5,371,014; 5,478,744; 5,179,022; 5,565,346; 5,484,956; 5,508,468; 5,538,877; 5,554,798; 5,489,520; 5,510,318; 5,204,253; 5,405,765; EP Nos. 267,159; 604,662; 672,752; 442,174; 486,233; 486,234; 539,563; 674,725; and, International Patent Application Publication Nos. WO 91/02071 and WO 95/06128, the entirety of each of which is hereby incorporated herein by this reference.

H Styrene Production

In some embodiments, the production of styrene by the transformed or recombinant host cells of the subject technology can be enhanced by exposing said cells to autoinducer molecules. Autoinducer molecules can be supplied to the cell in a culture medium. Autoinducer molecules may be supplied directly to the culture medium. Alternatively, the ethanologenic cells can be co-cultured with an autoinducer producing bacterial cell. Exemplary autoinducers include acylated homoserine lactones, furanosyl borate diester.

A recombinant host cell transformed or transfected with an expression vector of the subject technology, comprising the polynucleotides of the subject technology as described previously, can be used to produce (i.e., express) a styrene at an increased rate of production relative to a non-transformed or transfected cell.

In an embodiment, the subject technology comprises an expression system for the large scale production of styrene, utilizing a nucleic acid coding sequence of the subjection technology, encoding a PAL and an FDC protein.

In an embodiment, the recombinant host cells of the subject technology are grown or cultured under a condition suitable for production of styrene. As a rule, the styrene producing cells of the subject technology are grown in a liquid medium comprising a carbon source, usually in the form of sugars, a nitrogen source, usually in the form of organic nitrogen sources such as yeast extract or salts such as ammonium sulfate, trace elements such as salts of iron, manganese, magnesium, calcium, and/or zinc and, if appropriate, vitamins, at temperatures of between about 0° C. to about 100° C., or between about 10° C. to about 60° C., or between about 10° C. to about 20° C., or at about 16° C., while gassing in oxygen.

The pH of the liquid medium can either be kept constant, that is to say regulated during the culturing period, or not. The cultures can be grown batchwise, semi-batchwise or continuously. Nutrients can be provided at the beginning of the fermentation or fed in semi-continuously or continuously. The products produced can be isolated from the organisms as described above by processes known to the skilled worker, for example by extraction, distillation, crystallization, if appropriate precipitation with salt, and/or chromatography. To this end, the host cells can advantageously be disrupted beforehand. In this process, the pH value is advantageously kept between pH 4 and 12, or between pH 6 and 9, or between pH 7 and 8.

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981; the entirety of which is hereby incorporated herein by reference).

As described above, these media which can be employed in accordance with the subject technology usually comprise one or more carbon sources, nitrogen sources, inorganic salts, vitamins and/or trace elements.

In an embodiment, the carbon sources for use in the culture media of the host cells comprise sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

In an embodiment, the inorganic salt compounds that are present in the culture media comprise at least one of the chloride, phosphorus and sulfate salts of calcium, magnesium, sodium, cobalt, molybdenum, potassium, manganese, zinc, copper or iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing derivatives of styrene.

Phosphoric acid, potassium dihydrogenphosphate or dipotassium hydrogenphosphate or the corresponding sodium-containing salts may be used as sources of phosphorus.

Other components that may be added to the culture medium in order to keep the metal ions in solution comprise dihydroxyphenols such as catechol or protocatechuate and organic acids such as citric acid.

The fermentation media used according to the subject technology for culturing the host cells of the subject technology usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73; the entirety of which is hereby incorporated herein by reference). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 10° C. and 45° C. In an embodiment, the culture temperature is kept at a temperature between about 10° C. to about 40° C., or between about 10° C. to about 20° C., or between about 15° C. to about 20° C., or at about 16° C. or between about 25° C. to about 37° C., or between about 35° C. to about 37° C., or at about 37° C., and may be kept constant or may be altered during the culture period.

In some embodiments, the pH of the culture medium is kept in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of vector it is possible to add to the medium suitable substances having a selective effect, for example antibiotics. Aerobic conditions are maintained by introducing oxygen or oxygen-containing gas mixtures such as, for example, ambient air into the culture. The temperature of the culture is normally 10° C. to 45° C. The culture is continued until formation of the desired product is at a maximum. This aim is normally achieved within 10 to 160 hours.

In an embodiment, the fermentation broth can be processed further to remove the biomass (e.g., styrene or cinnamic acid) produced in the media. The biomass may be removed completely or partially from the fermentation broth by separation methods such as, for example, centrifugation, filtration, decanting or a combination of these methods or be left completely in said broth.

However, the fermentation broth can also be thickened or concentrated without separating the cells, using known methods such as, for example, with the aid of a rotary evaporator, thin-film evaporator, falling-film evaporator, by reverse osmosis or by nanofiltration.

In an embodiment, the recombinant host cells of the subject technology can be cultured using a batch fermentation, particularly when large scale production of styrene using an expression system of the subject technology is required. Alternatively, a fed batch and/or continuous culture can be used to generate a yield of styrene from host cells transformed with an expression system of the subject technology.

In an embodiment, the recombinant host cells of the subject technology can be cultured in aerobic or anaerobic conditions. In aerobic conditions, preferably, oxygen is continuously removed from the culture medium, by for example, the addition of reductants or oxygen scavengers, or, by purging the reaction medium with neutral gases.

Techniques known in the art for the large scale culture of host cells are disclosed in for example, Bailey and 011 is (1986) Biochemical Engineering Fundamentals, McGraw-Hill, Singapore; or Shuler (2001) Bioprocess Engineering: Basic Concepts, Prentice Hall. All such techniques are hereby incorporated herein by reference.

The host cells of the subject technology can be cultured in a vessel, for example a bioreactor. Bioreactors, for example fermentors, are vessels that comprise cells or enzymes and typically are used for the production of molecules on an industrial scale. The molecules can be recombinant proteins or compounds that are produced by the cells contained in the vessel or via enzyme reactions that are completed in the reaction vessel. Typically, cell based bioreactors comprise the cells of interest and include all the nutrients and/or co-factors necessary to carry out the reactions.

Where commercial production of styrene is desired a variety of fermentation methodologies may be applied. For example, large scale production may be effected by both batch or continuous fermentation.

A classical batch fermentation is a closed system where the composition of the media is set at the beginning of the fermentation and not subject to artificial alterations during the fermentation. Thus, at the beginning of the fermentation the medium is inoculated with the desired microorganism or microorganisms and fermentation is permitted to occur adding nothing to the system. Typically, however, the concentration of the carbon source in a “batch” fermentation is limited and attempts are often made at controlling factors such as pH and oxygen concentration. In batch systems the metabolite and biomass compositions of the system change constantly up to the time the fermentation is stopped. Within batch cultures cells moderate through a static lag phase to a high growth log phase and finally to a stationary phase where growth rate is diminished or halted. If untreated, cells in the stationary phase will eventually die. Cells in the log phase generally are responsible for the bulk of production of end product or intermediate.

A variation on the standard batch system is the Fed-Batch system. Fed-Batch fermentation processes are also suitable in the subject technology and comprise a typical batch system with the exception that the substrate is added in increments as the fermentation progresses. Fed-Batch systems are useful when catabolite repression is apt to inhibit the metabolism of the cells and where it is desirable to have limited amounts of substrate in the medium. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch fermentations are common and well known in the art and examples may be found in Brock, T. D.; Biotechnology: A Textbook of Industrial Microbiology, 2nd ed.; Sinauer Associates: Sunderland, Mass., 1989; or Deshpande, M. V. Appl. Biochem. Biotechnol. 36:227, (1992), the entirety of each of which is hereby incorporated herein by reference.

Commercial production of styrene may also be accomplished with continuous fermentation. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in their log phase of growth. Continuous fermentation allows for modulation of any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by the medium turbidity, is kept constant. Continuous systems strive to maintain steady state growth conditions and thus the cell loss due to the medium removal must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

I Production of Downstream Styrene Derivatives

In some embodiments of the subject technology, the styrene produced in the host cells can further undergo enzymatic reaction and be converted to a downstream derivative of styrene such as toluene, xylene, polystyrene, ABS, styrene-butadiene (SBR) rubber, styrene-butadiene latex, SIS (styrene-isoprene-styrene), S-EB-S (styrene-ethylene/butylene-styrene), styrene-divinylbenzene (S-DVB), styrene-acrylonitrile resin (SAN) and unsaturated polyesters and the like. Thus, in some embodiments the recombinant host cells of the subject technology can convert a percentage of the styrene produced to a downstream derivative of styrene. In a related embodiment, the host cell can convert at least 5% of the styrene to a downstream derivative. In another related embodiment, the host cell can convert about 5% to 15% of the styrene to a downstream derivative, or can convert about 10% to 25% of the styrene to a downstream derivative, or can convert about 20% to 35% of the styrene to a downstream derivative, or can convert about 30% to 45% of the styrene to a downstream derivative, or can convert about 40% to 55% of the styrene to a downstream derivative, or can convert about 50% to 65% of the styrene to a downstream derivative, or can convert about 60% to 75% of the styrene to a downstream derivative, or can convert about 70% to 85% of the styrene to a downstream derivative, or can convert about 80% to 95% of the styrene to a downstream derivative.

The practice of the subject technology can employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Polynucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986; the entirety of each of which is incorporated herein by reference.

The subject technology is further illustrated by the following specific but non-limiting examples. The following examples may include compilations of data that are representative of data gathered at various times during the course of development and experimentation related to the subject technology.

### EXAMPLES

The subject technology is further defined in the following Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### Example 1

### Cloning of yeast PAD1 gene

To clone the yeast PAD1 polynucleotide, the genomic DNA of yeast Saccharomyces cerevisiae was isolated by a protocol of Markus Ralser. Briefly, 1.5 ml of liquid culture of yeast grown for 20-24 h at 30° C. in YPD (1% yeast extract, 2% peptone, 2% dextrose) was transferred into a microcentrifuge tube. Cells were spun down by centrifugation at 20,000×g for 1-5 minutes. 200 μl of Harju-buffer was added to the tubes. Tubes were immersed in a dry ice-ethanol bath for 2 minutes, then were transferred to in a 95° C. water bath for 1 minute. The last two steps were repeated. Tubes were vortexed for 30 seconds. 200 μl of chloroform was added to each tube and tubes were vortexed for 2 minutes. Tubes were spun for 3 minutes at room temperature at 20,000×g. The upper aqueous phase was transferred to a microcentrifuge tube containing 400 μl ice-cold 100% ethanol. Contents were mixed by inversion or gentle vortexing. Tubes were incubated at room temperature for 5 minutes. Alternatively, DNA could be precipitated at −20° C. to increase yield. Tubes were centrifuged for 5 minutes at room temperature at 20,000×g. The supernatant was removed with a pulled Pasteur pipette by vacuum aspiration. Pellets were washed with 0.5 ml 70% ethanol. Tubes were centrifuged for 5 minutes at room temperature at 20,000×g. Supernatant was removed and the pellets were air dried at room temperature or for 5 minutes at 60° C. in a vacuum dryer. Dried pellets were resuspended in 25-50 μl TE (pH 8.0)] or water. Samples obtained directly from plates were resuspended in a 10 μl volume, because the yield was smaller. (See also www.protocol-online.org/prot/Protocols/Quick-and-Easy-Isolation-of-Genomic-DNA-from-Yeast-3451.html).

The yeast strain used was Clontech Y2HGold cells. The primers were designed based on published genome sequence (YPAD-F (SEQ ID NO:13): ATGCTCCTATTTCCAAGAAGAACTA, YPAD-R (SEQ ID NO:14): TTACTTGCTTTTTATTCCTTCCCAA). The PCR was carried out using an Eppendorf Master Gradient thermocycler, under the condition as initial denaturation: 94° C. for 2 minutes, 30 cycles of: denature: 94° C. for 30 seconds, anneal: 55° C. for 30 seconds, and extend: 68° C. for 1 minute, and finally a 10 min of extension at 68° C. followed using the Invitrogen Platinum® Taq DNA Polymerase High Fidelity according to manufacturer's introduction (Invitrogn, Carlsbad, Calif.), (See: tools.invitrogen.com/content/sfs/manuals/platinumtaqhifi_pps.pdf, the entirety of which is hereby incorporated herein by reference, for more detail).

The resulting 0.729 kb PCR fragment was purified from 1% agarose gel, cloned into PCR8/GW/TOPO vector (Invitrogen), and fully sequenced using primers M13 (−20) forward and M13 reverse by Genewitz, (South Plainfield, N.J.). The resulting sequence is listed as SEQ ID NO:9. When this sequence is compared to the published yeast genome sequence, we found 0 number of mismatches.

This yeast PAD1 sequence was sub-cloned into a yeast expression vector and an E. coli expression vector. For the yeast expression vector, yeast PAD1 in PCR8 was cloned into yeast Gateway vector 304GPD-ccdB (Trp) through LR reaction. The 304GPD-ccdB (Trp) was published by Simon Alberti, Aaron D. Gitler, and Susan Lindquist in “A suite of Gateway cloning vectors for high-throughput genetic analysis in Saccharomyces cerevisiae. Yeast 2007; 24: 913-919.” The resulting vector is named 304GPD-PAD1 (Trp).

For the E. coli expression vector, yeast PAD1 was cloned into E. coli Gateway vector pDEST17 (Invitrogen) through LR reaction, resulting in plasmid pDEST17-PAD1.

### Example 2

### Cloning of Yeast FDC Gene

To clone the yeast FDC polynucleotide, the genomic DNA of yeast S. cerevisiae was isolated as above. The primers were designed based on published genome sequence (YFDC-F (SEQ ID NO:11): ATGAGGAAGCTAAATCCAGCTTTAGA, and YFDC-R (SEQ ID NO:12): TTATTTATATCCGTACCTTTTCCAA). The PCR was carried out as described in Example 1, except the PCR condition as 30 cycles of: denature: 94° C. for 30 seconds, anneal: 55° C. for 30 seconds, and extend: 68° C. for 2 minute.

The resulting 1.512 kb PCR fragment was purified and sub-cloned into PCR8/GW/TOPO vector (Invitrogen). The resulting vector was fully sequenced using primers M13 (−20) forward and M13 reverse by Genewitz, (South Plainfield, N.J.). The resulting sequence is listed as SEQ ID NO:7. When this sequence is compared to the published yeast genome sequence, we found 0 number of mismatches.

This yeast FDC sequence was sub-cloned into a yeast expression vector and an E. coli expression vector. For the yeast expression vector, Yeast FDC in PCR8 was cloned into yeast Gateway vector 303GPD-ccdB (His) through LR reaction. The 303GPD-ccdB (His) was published by Simon Alberti, Aaron D. Gitler, and Susan Lindquist previously. The resulting vector is named 303GPD-FDC.

For the E. coli expression vector, yeast FDC polynucleotide was cloned into E. coli Gateway vector pDEST17 (Invitrogen) through LR reaction, resulting in plasmid pDEST17-FDC.

### Example 3

### Cloning of Arabidopsis PAL Genes

To obtain a functional PAL enzyme, full-length cDNAs of Arabidopsis PAL1 (At2g37040), PAL2 (At3g53260) and PAL4 (At3g10340) were purchased from Arabidopsis Biological Resource Center (ABRC) at the Ohio State University under the stock numbers G10120 (in pENTR223.1 vector), G12256 (in pENTR223.1 vector), and U24927 (in pENTR/D-TOPO vector), respectively. These genes have been functional characterized and published previously. (Fiona C Cochrane, Laurence B Davin and Norman G Lewisin “The Arabidopsis phenylalanine ammonia lyase gene family: kinetic characterization of the four PAL isoforms. Phytochemistry, Volume 65, Issue 11, June 2004, Pages 1557-1564.”)

Arabidopsis PAL1, PAL2, and PAL4 in pENTR223.1 or pENTR/D-TOPO were cloned into yeast Gateway vector 305GPD-ccdB (Leu) through LR reaction, resulting 305GPD-PAL1, 305GPD-PAL2 and 305GPD-PAL4, respectively.

### Example 4

### Methods for Sample Preparation and Product Analysis

0.8 ml of well-suspended cultures was taken out of the culture tube and 0.4 ml of ethyl acetate was added. The mixture was votexed at the maximum speed for half an hour and centrifuged at 15,000 rpm for 10 min at room temperature.

The extracted samples were analyzed by HPLC. HPLC was performed with an Agilent 1100 Series HPLC system using a SymmetryShield C18 column (Waters, 150×3.9 mm). The mobile phase consisted of solvent A (0.1% trifluoroacetic acid) and solvent B (acetonitrile). The gradient elution procedure was as follows: 0-5 min, 0% of B; 5-35 min, a linear gradient from 0% to 90% of B; 35-38 min, 90% of B; 38-40 min, 0% of B. The flow rate was 1.0 ml/min. The injection volume was 30 μl. Peaks for substrate and products areas were monitored at 260 nm. With this method, the retention times for cinnamic acid, p-coumaric acid, ferulic acid, styrene, 4-hydroxystyrene, and 4-vinylguaiacol (4-VG) were 21.5, 17.7, 17.7, 27.8, 22.3 and 22.1 min, respectively.

### Example 5

### Functional Expression of Full Length PAD1 in Yeast and E. Coli

To examine the activity of yeast PAD1, the cloned PAD1 polynucleotide was cloned into an expression vector, resulting in 304GPD-PAD1 as described above. The vector was transformed into Wat11 yeast cells. Wat11 has been published previously. Yeast transformation was carried out based on the protocol published by Dz-Chi Chen, Bei-Chang Yang and Tsong-Teh Kuoin “One-step transformation of yeast in stationary phase” CURRENT GENETICS, Volume: 21, Issue: 1, Pages: 83-84, Published: January 1992.

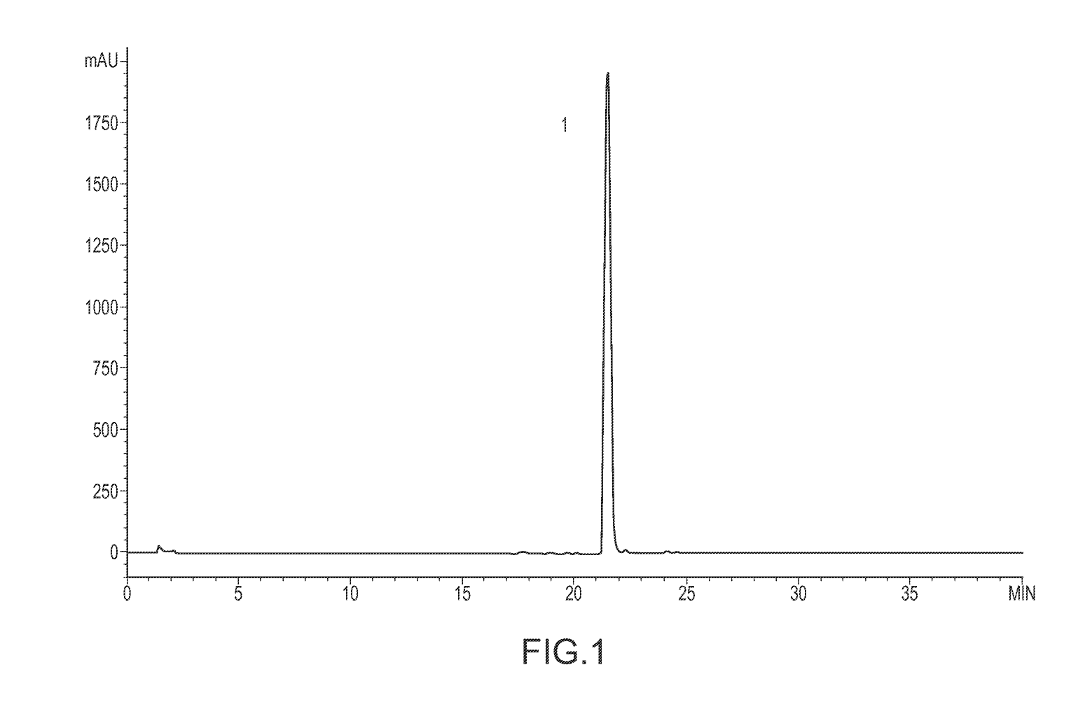

After transformation, 100 μl of the transgenic yeast culture was spread on SD-trp medium (Clontech, Mountain View, Calif.) and incubated at 30° C. for 3 days. Single colonies were picked and used to inoculate 10 ml of liquid SD-Trp medium. They were grown under 30° C. temperature and shaker speed 250 rpm for two days. 2 mM cinnamic acid was added to 2 d-old yeast culture and the products were extracted with ethyl acetate after 8 hours and analyzed by HPLC. As shown in FIG. 1, no styrene can be observed in the HPLC profile.

Plasmid pDEST17-PAD1 was transformed into E. coli strain BL21AI competent cells (Invitrogen). An overnight culture (2 ml) was inoculated into 40 ml of LB medium supplemented with 100 g/ml ampicillin. Bacteria were grown at 37° C. in a shaker at 250 rpm for 3 h and then 0.2% (w/v) arabinose was added to the culture, followed by the addition of 2 mM of cinnamic acid. The induced culture was further incubated at 37° C. and samples were taken at different time points for the analysis.

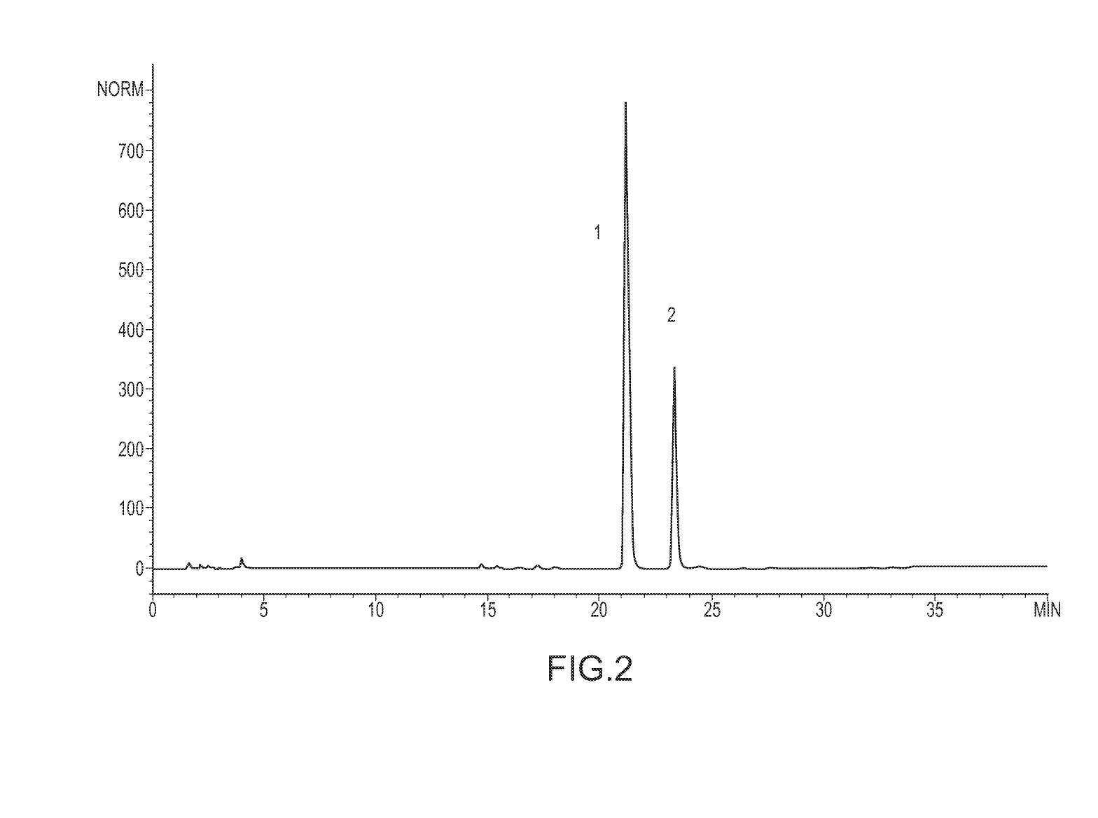

Once again, no styrene production was observed. See FIG. 2. We observed a unknown peak in E. coli fed with cinnamic acid but it's retention time and UV-Vis spectrum did not match those of styrene.

Accordingly, we concluded that the full-length yeast PAD1 cannot be used to produce styrene under the conditions set forth in this Example.

### Example 6

### Functional Expression of the Truncated Yeast PAD1

We used three targeting predicting software to predict the subcellular localization of yeast PAD1. All of them predicted that PAD1 can be localized to mitochondria in yeast cells.

Method 1: PAD sub-cellular localization prediction by TargetP.1.1 (www.cbs.dtu.dk/services/TargetP/). TargetP 1.1 predicts the subcellular location of eukaryotic proteins. The location assignment is based on the predicted presence of any of the N-terminal presequences: chloroplast transit peptide (cTP), mitochondrial targeting peptide (mTP) or secretory pathway signal peptide (SP).

TargetP 1.1 Server - prediction results

Technical University of Denmark
Number of query sequences: 1
Cleavage site predictions not included.
Using NON-PLANT networks.
### targetp v1.1 prediction results ################################
##
Number of query sequences: 1
Cleavage site predictions not included.
Using NON-PLANT networks.

| Name | Len | mTP | SP | Other | Loc | RC |
|---|---|---|---|---|---|---|
| Sequence | 242 | 0.795 | 0.066 | 0.104 | M | 2 |
| Cutoff | | 0.000 | 0.000 | 0.000 | | |

Method 2. PAD sub-cellular localization prediction by WoLF PSORT (wolfpsort.org).

k used for kNN is: 27
queryProtein details mito: 12.0, plas: 8.0, cyto: 2.0, nucl: 1.0, extr: 1.0,
pero: 1.0

Method 3. PAD sub-cellular localization prediction by SubLoc v1.0 (www.bioinfo.tsinghua.edu.cn/SubLoc/eu_predict.htm)

Results predicted by SubLoc v1.0
-- for Eukaryotic sequence --

Protein Sequence Information
Sequence Name: Unknown Sequence
Sequence Length: 242
the number of non-standard amino acids: 0
Prediction of Subcellular Localization by SubLoc
Predicted Location: Mitochondrial
Reliability Index: RI = 2; Expected Accurcy = 74%

To obtain a functional expression in yeast cytosol, we truncated the PAD1 gene to remove the mitochondria targeting sequence (the first 31 amino acid at the N-terminus) and named this gene PAD1-TP31. The truncated cDNA was 636 by (SEQ ID NO:15).

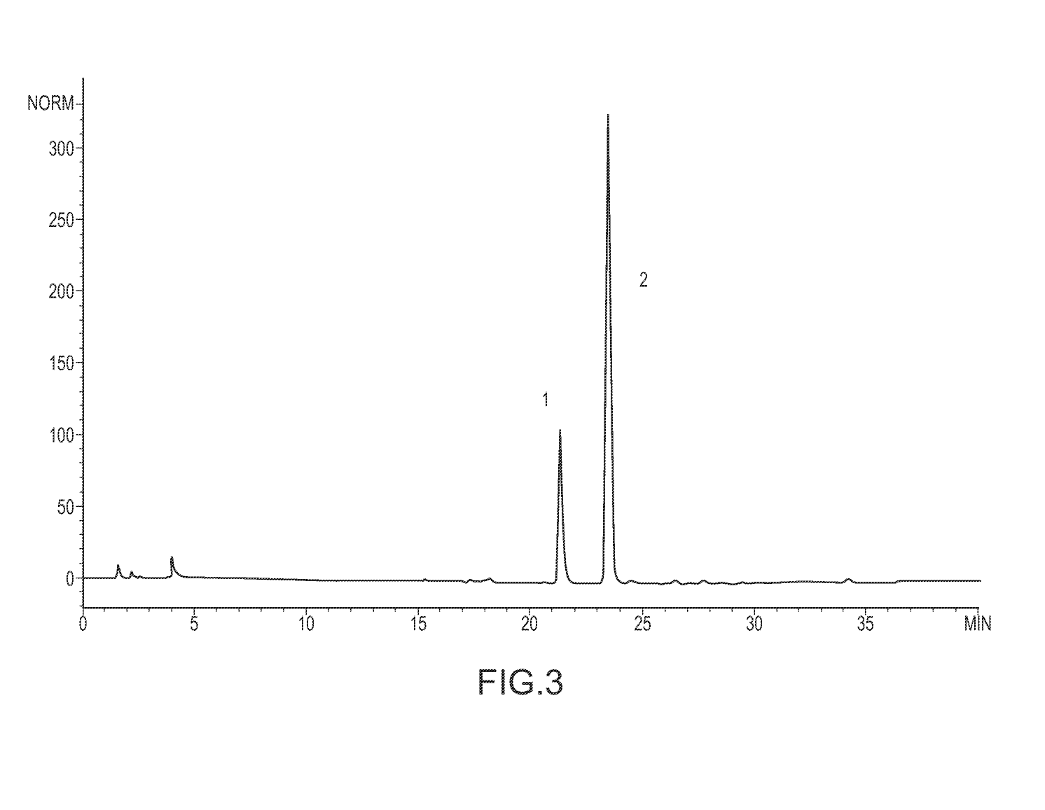

To facilitate the functional express PAD1-TP31 truncated protein, we added one ATG at the N-terminus of the truncated sequence. PAD1-TP31 was first cloned into PCR8/TOPO/GW vector and swapped into pDEST17 and 304GPD-ccdB (Trp) vectors through LR reaction for its expression in E. coli and yeast, respectively. FIG. 2 is the HPLC profile of E. coli expressing full-length PAD1 gene after being fed with 2 mM of cinnamic acid. As shown in this figure no styrene was produced. FIG. 3 is the HPLC profile of E. coli expressing truncated PAD1 gene after being fed with 1 mM of cinnamic acid. Again, E. coli could not produce styrene.

Taken together, we concluded that over-expression of either the full-length PAD1, or the truncated version of PAD1 that lacks the mitochondria sequence is also incapable of making styrene in yeast and E. coli cells under conditions specified here.

### Example 7

### Functional Expression of Yeast FDC

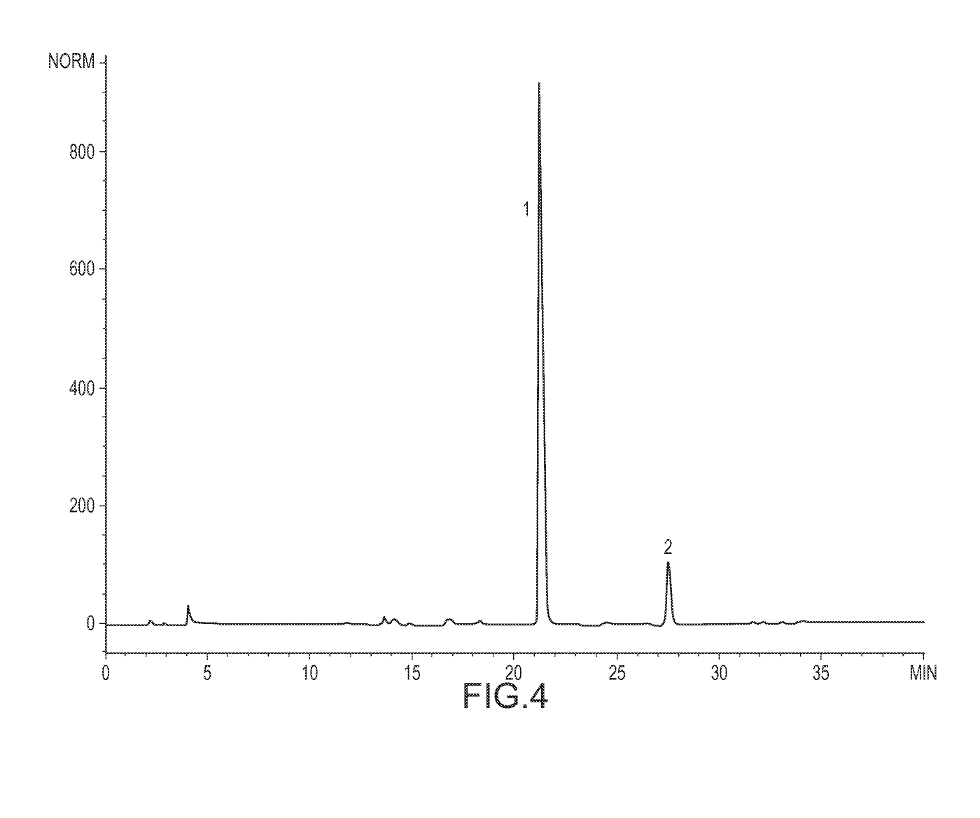

To examine the activity of yeast FDC, the cloned FDC gene was cloned into an expression vector, resulting in 303GPD-FDC as described above. The vector was transformed into Wat11 yeast cells, and analyzed as described in Example 4, except that FDC in 303GPD-ccdB (His) in WAT11 were cultured in SD-His medium. As shown in FIG. 4, when cinnemic acid was fed to the culture media, we could observe the production of styrene (in FIG. 4, the retention times for cinnamic acid and styrene are 21.5 and 27.7 min, respectively).

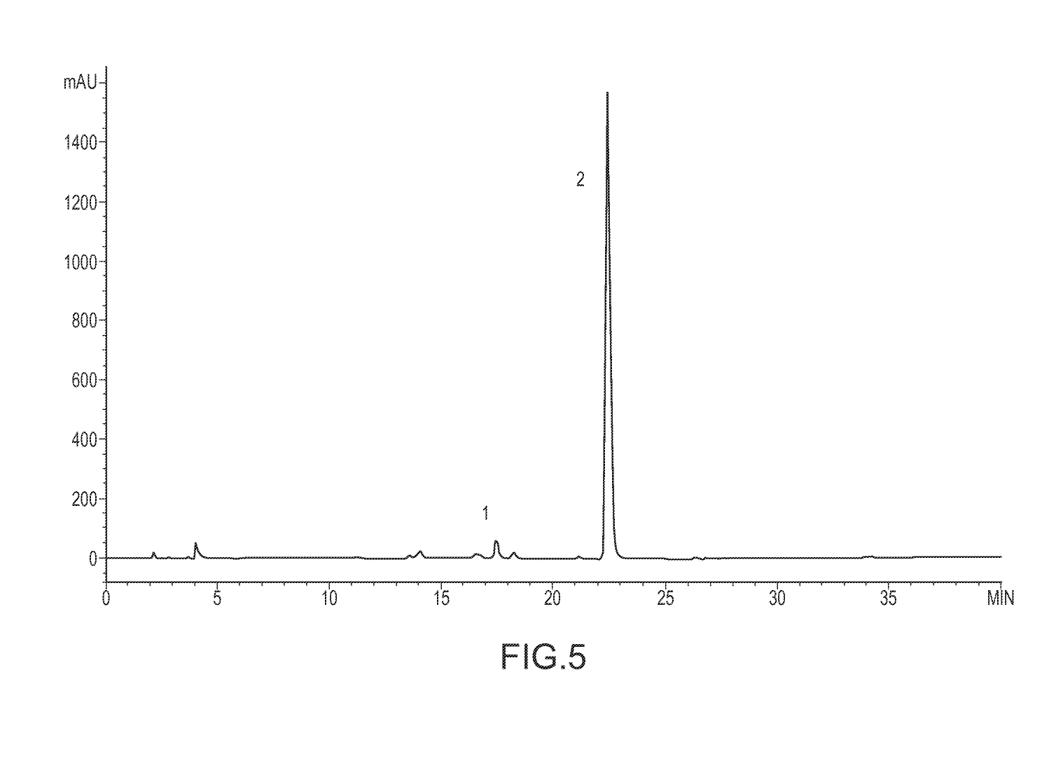

As shown in FIG. 5, when p-coumaric acid was fed to the culture media, we could observe the production of 4-hydroxystyrene.

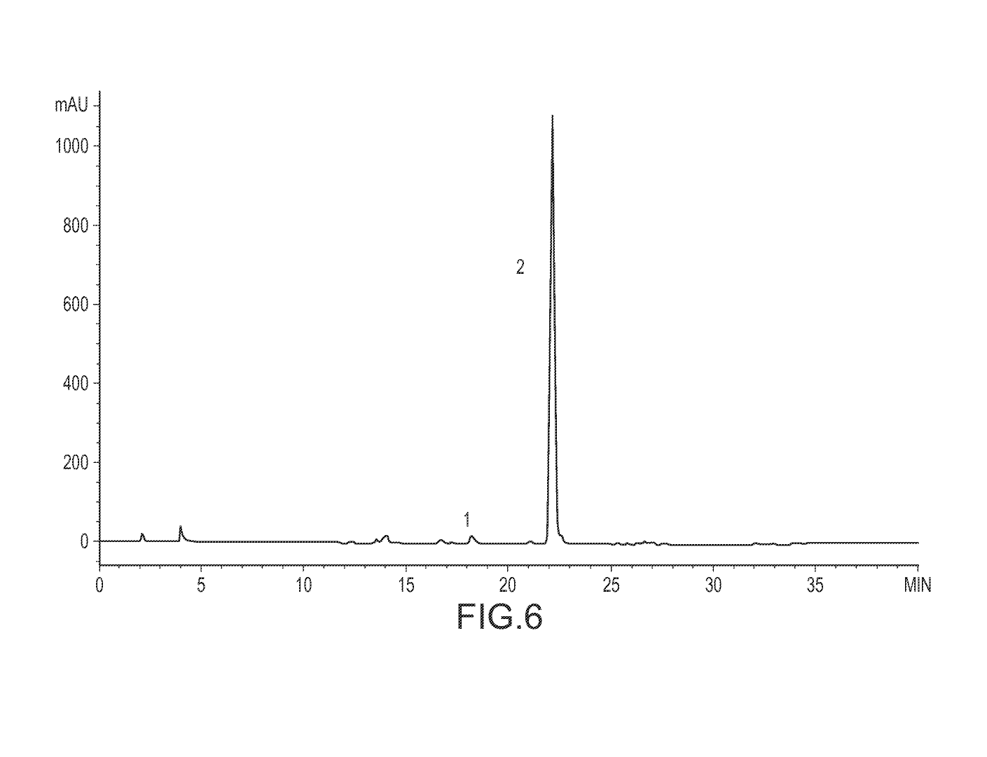

As shown in FIG. 6, when ferulic acid was fed to the culture media, we could observe the production of 4-VG.

Therefore, production of styrene, 4-hydroxystyrene and 4-vinylguaiacol was observed in yeast Wat11 culture carrying FDC in 303GPD-ccdB (His) vector upon the feeding of cinnamic acid, coumaric acid and ferulic acid, respectively.

In E. coli, pDEST17-FDC was transformed into E. coli strain BL21AI competent cells (Invitrogen). An overnight culture (2 ml) was inoculated into 40 ml of LB medium supplemented with 100 g/ml ampicillin. Bacteria were grown at 37° C. in a shaker at 250 rpm for 3 h and then 0.2% (w/v) arabinose was added to the culture, followed by the addition of 2 mM of cinnamic acid. The induced culture was further incubated at 37° C. and samples were taken at different time points for the analysis.

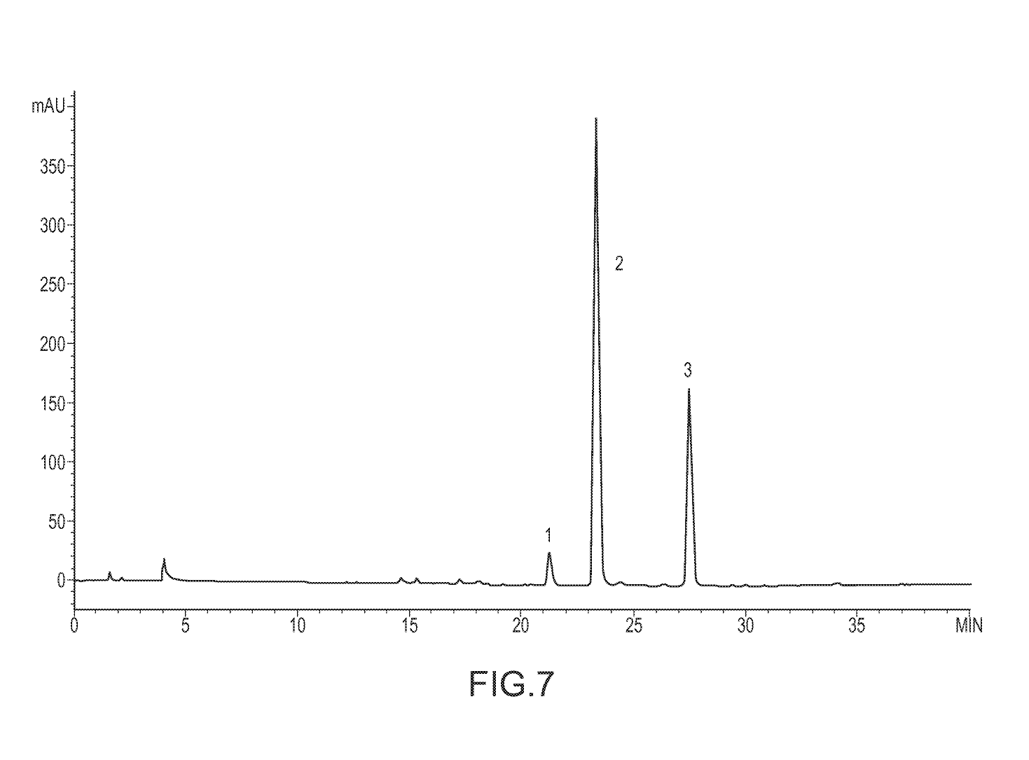

Production of styrene was observed in E. coli culture carrying FDC in pDEST17 vector upon the feeding of cinnamic acid. See FIG. 7. This result indicated that FDC, independent of phenylacrylic acid decarboxylase (PAD1), can convert cinnamic acid to styrene.

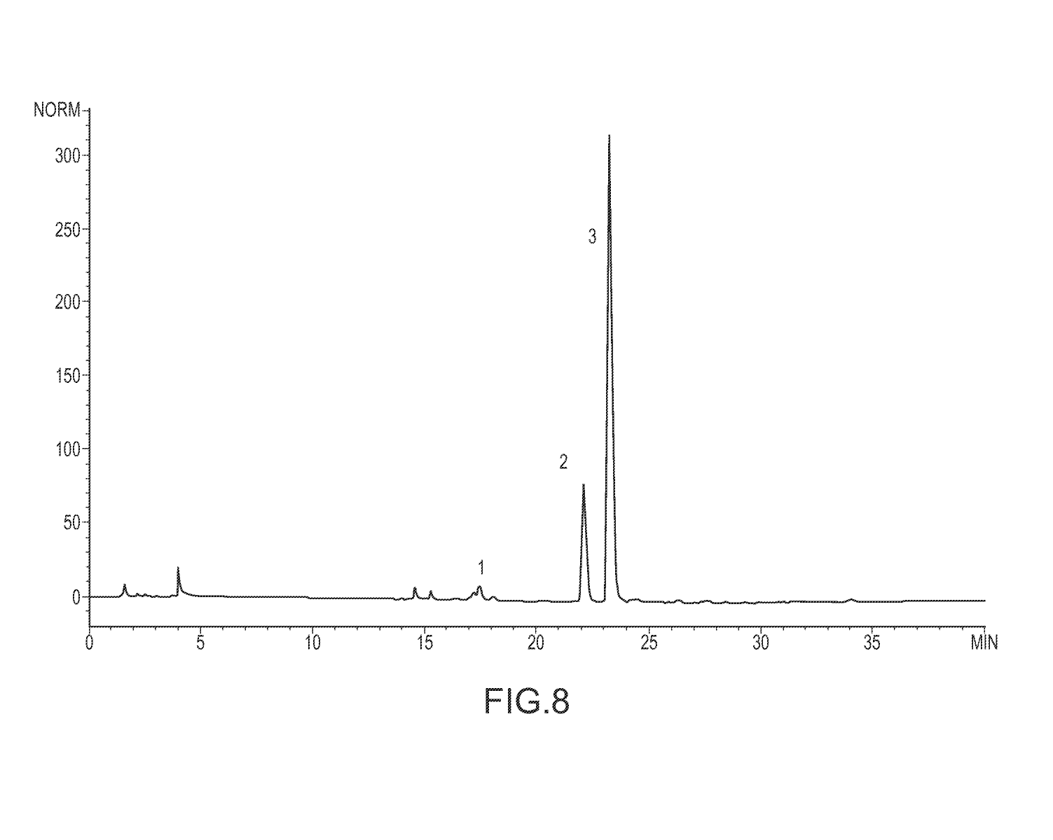

Production of 4-vinylguaiacol (4-VG) was also observed in E. coli culture carrying FDC in pDEST17 vector upon the feeding of ferulic acid to the E. coli. See FIG. 8.

### Example 8

### Co-Expression of Arabidopsis PAL and Yeast FDC Produced Styrene in Yeast in the Absence of Feeding

To study the possibility of de novo biosynthesis of styrene in yeast cells, we co-transformed Arabidopsis PAL genes and FDC gene in yeast.

The cloning of the PAL genes was described in Example 3. We transformed the AtPAL1, AtPAL2, or AtPAL4 with FDC to see which PAL can be used to couple with FDC to produce styrene.

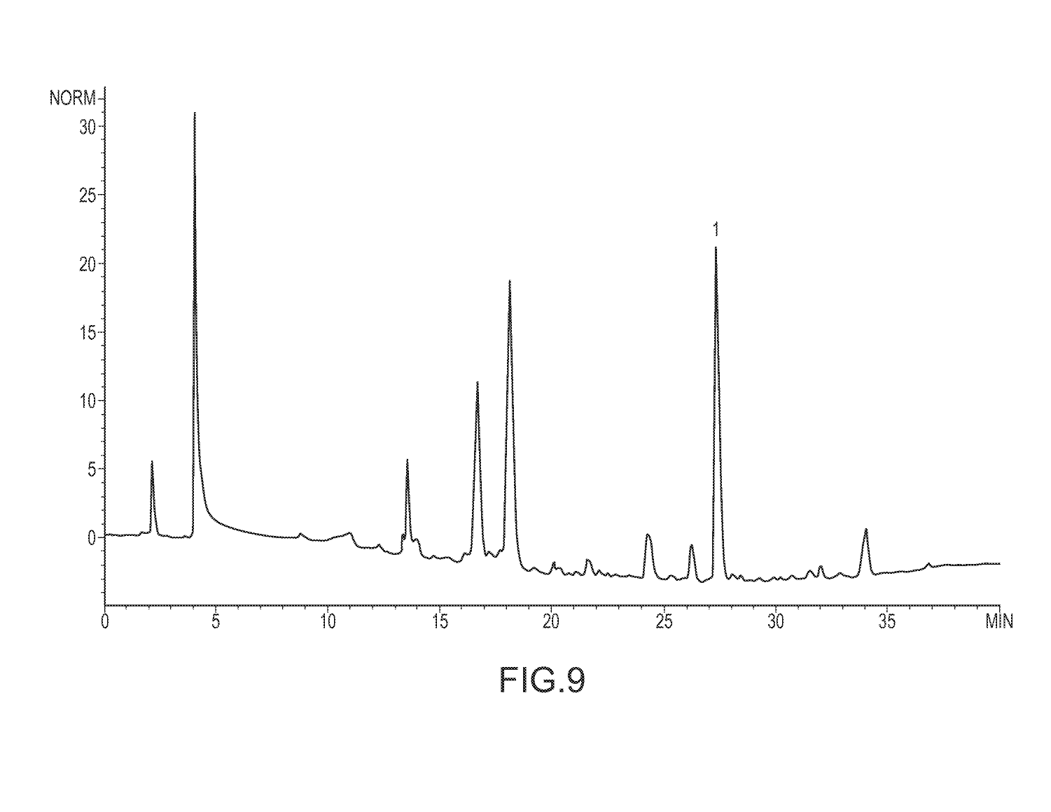

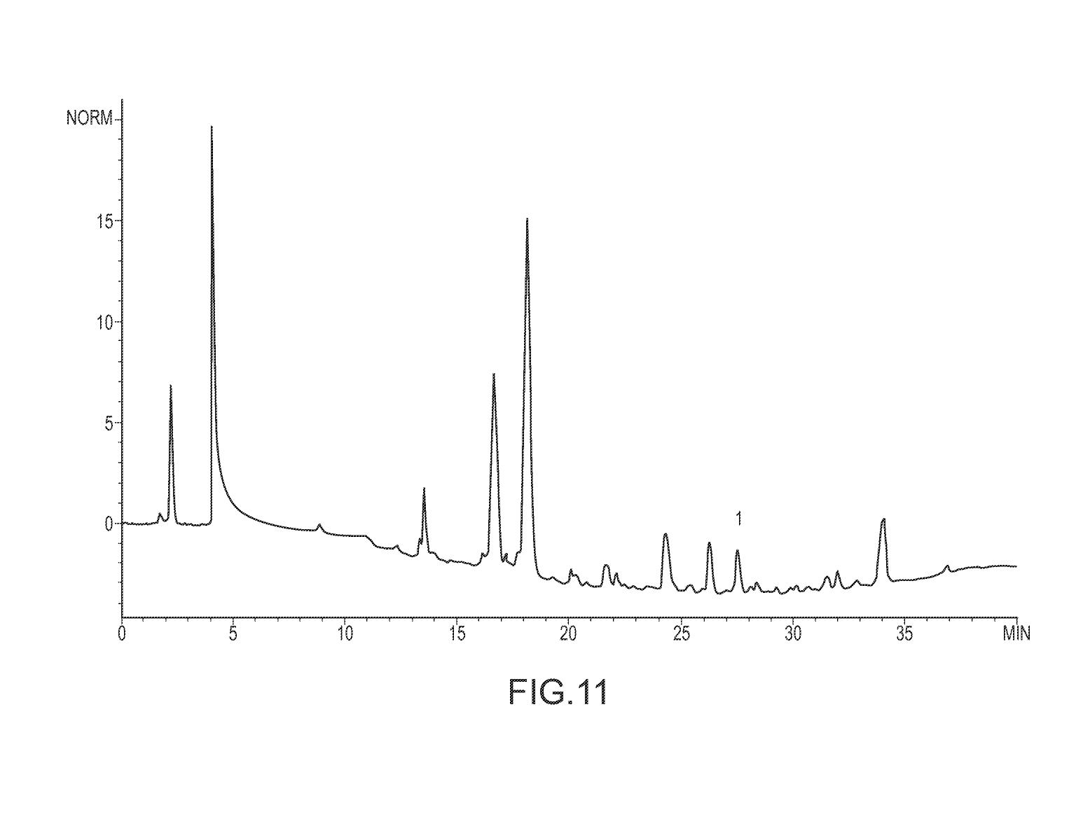

As shown in FIGS. 9, 10 and 11, all of these PAL-FDC co-transformants could produce styrene de novo and in the absence of any feeding. However, PAL2-FDC combination gave the highest production.

### Example 9

### Interaction Between PAD1 and FDC in a Yeast Two Hybrid System

Nobuhiko Mukai, et al. reported that both PAD1 and FDC are essential for the production of styrene upon the feeding of cinnamic acid (Journal of Bioscience and Bioengineering Volume 109, Issue 6, June 2010, Pages 564-569). So it has been suggested that these protein may form hetero-oligomeric structures in vivo (Plumridge et al., Fungal Genetics and Biology Volume 47, Issue 8, August 2010, Pages 683-692). We used yeast-two-hybrid system to test this hypothesis. Yeast PAD1 and FDC were cloned into yeast two hybrid vectors pGBKT7-Gateway and pGADT7-Gateway respectively by LR reaction. Vectors pGBKT7-Gateway and pGADT7-Gateway are gifts from Dr. Brian P. Downes, Department of Biology, St Louis University (St Louis, Mo.). The resulting vectors were used to transform Clontech Y2HGold cells by one step transformation protocol as described in Example 4. 100 μl of the transformed cultures were spread on SD-Trp-Leu as well as SD-Trp-Leu-His plates and incubated at 30° C. for 3 days.

As shown in Table 2, All these transformants can grow on SD-Trp-Leu plates, indicating the transformation was successful. However, only the transformants with FDC-FDC combination can grow on SD-Trp-Leu-His plate. These results seem to suggest that FDC may form a homodimer and that PAD1 and FDC may not form an enzyme complex.

TABLE 2
Y2H assay of yeast PAD1 and FDC interaction.

| Bait vector | Prey vector | SD-Trp-Leu | SD-Trp-Leu-His |
|---|---|---|---|
| PAD1 | PAD1 | + | − |
| PAD1 | FDC | + | − |
| FDC | PAD1 | + | − |
| FDC | FDC | + | + |

### Example 10

### Other Genes that have Styrene Synthesis Activity

The two Aspergillus niger genes, AnPADA1 and AnOHBA1, are reported to be homologues of yeast PAD1 and FDC, respectively (Andrew Plumridge et al., The decarboxylation of the weak-acid preservative, sorbic acid, is encoded by linked genes in Aspergillus spp. Fungal Genetics and Biology Volume 47, Issue 8, August 2010, Pages 683-692; the entirety of which is hereby incorporated herein by reference). We set out to prove that the homologous genes of FDC in fungi could also produce styrene in our assay conditions.

We first synthesized AnPADA1 (SEQ ID NO:17) and AnOHBA1 (SEQ ID NO:18) genes through GenScript USA Inc. (Piscataway, N.J., www.genscript.com/) based their sequences in NCBI database (www.ncbi.nlm.nih.gov/nucleotide/) with GenBank numbers EF215454.1 and XM_{—}001390497.1, respectively.

The synthesized genes have 31 by and 30 by additional sequences at the 5′ and 3′ ends, respectively (Bold characters in the sequences) to facilitate Gateway cloning. The synthesized DNA sequence in pUC57 vector was obtained from GenScript and swapped into pDONR/Zeo vector though Gateway® BP reaction (Invitrogen). The resulting plasmids are named as pDONR-AnPADA1 and pDONR-AnohbA1, respectively.

pDONR-AnPADA1 and pDONR-AnOHBA1 are cloned into pDEST17 vector for their expression in E. coli. They are also cloned into 304GPD-ccdB (Trp) and 303GPD-ccdB (His) vectors, resulting 304GPD-AnPAD1 (Trp) and 303GPD-AnohbA1 (His), respectively.

### Example 11

### Comparison of PAD1, FDC, and OHBA1 Activities

E. coli cells containing FDC, PAD1, and OHBA1, a FDC-homologous clone from Aspergillus, were grown in LB+Amp medium, and 1-40 mM of cinnamic acid was fed as substrate to medium. Sample (0.5 ml) was taken various treatments, and extracted with 0.3 ml of ethyl acetate. The upper phase was used for the analysis of the product styrene, and the lower phase for the analysis of cinnamic acid left in the medium.

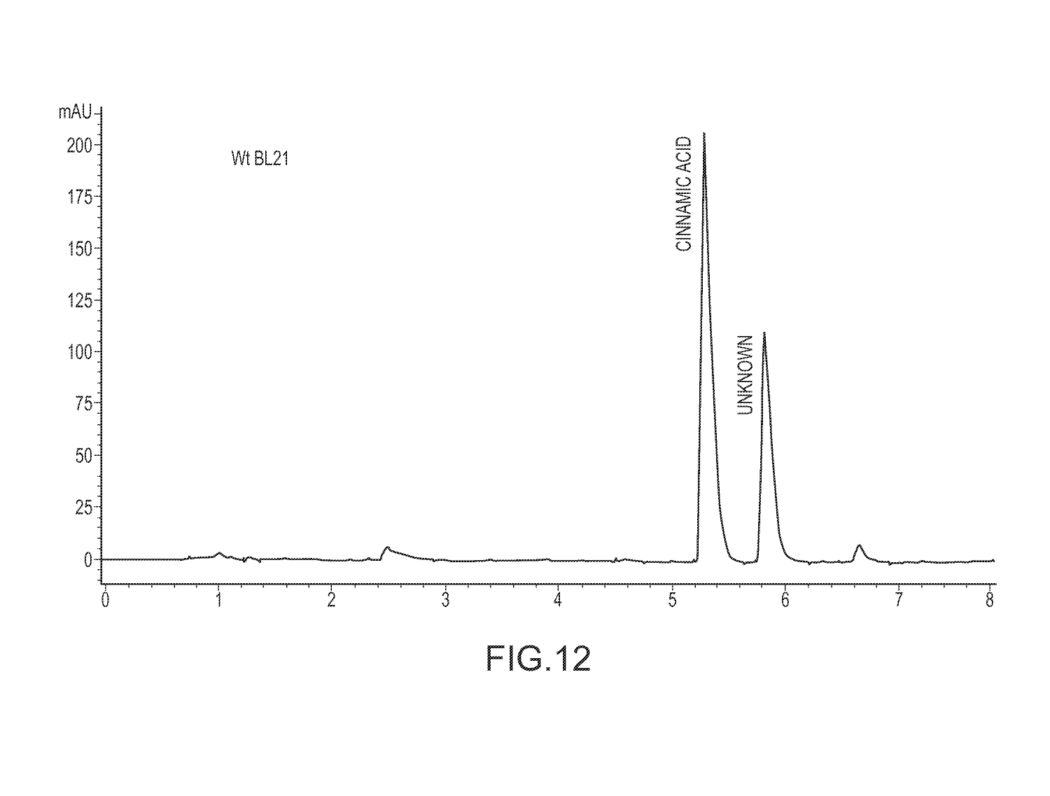

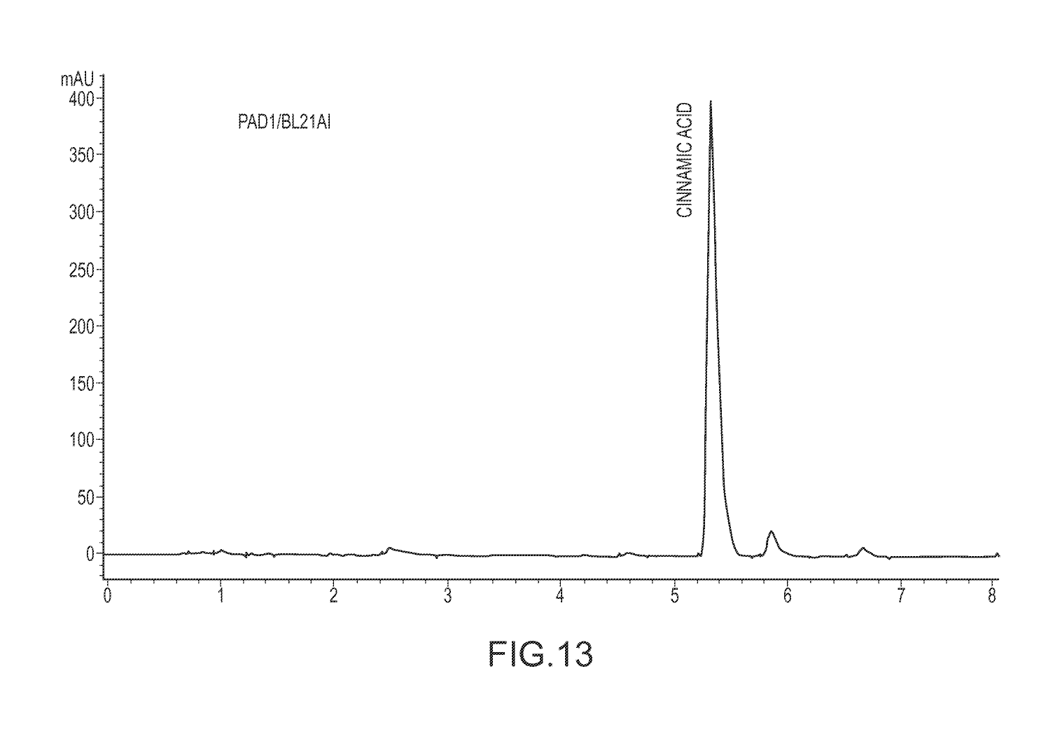

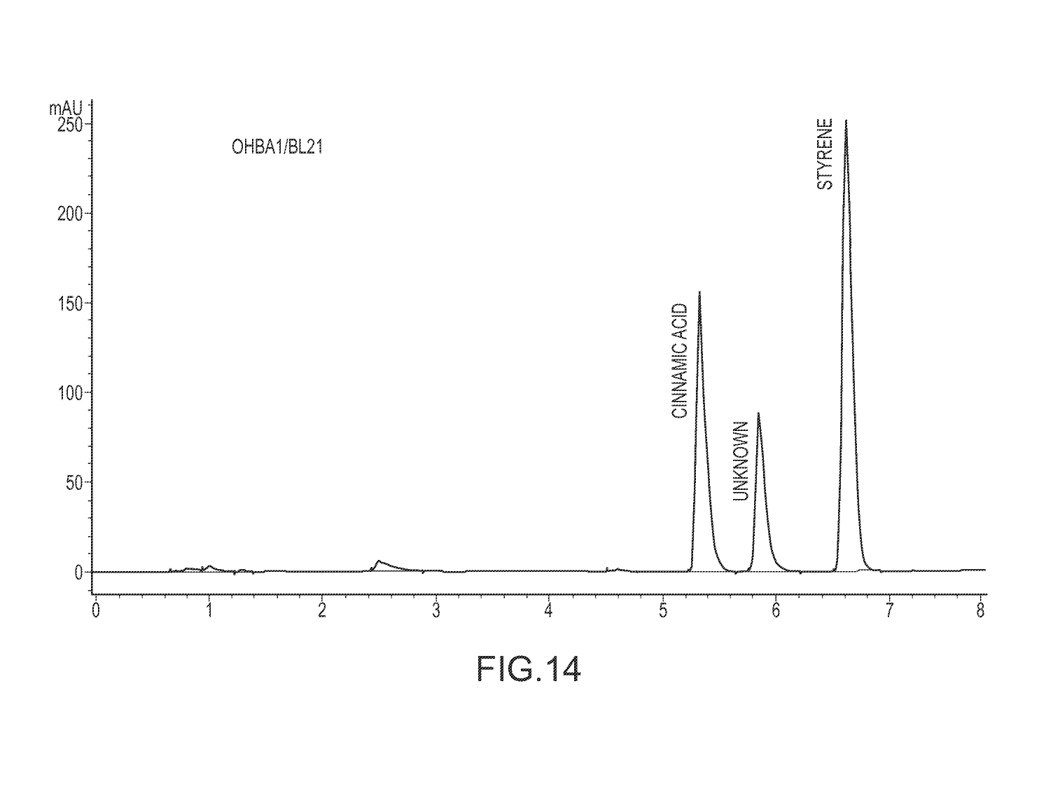

Firstly, the substrate 1-10 mM of cinnamic acid was tested in wild type E. coli. See FIG. 12. Then, E. coli cells carrying the PAD1 gene were fed with cinnamic acid but failed to produce any styrene. See FIG. 13. However, when E. Coli cells carrying OHBA1 gene were fed with cinnamic acid, they produced styrene. See FIG. 14. The highest styrene level (40.1 mg/L) was found in the medium containing 10 mM substrate. The corresponding reduction of substrate was observed (FIG. 14).

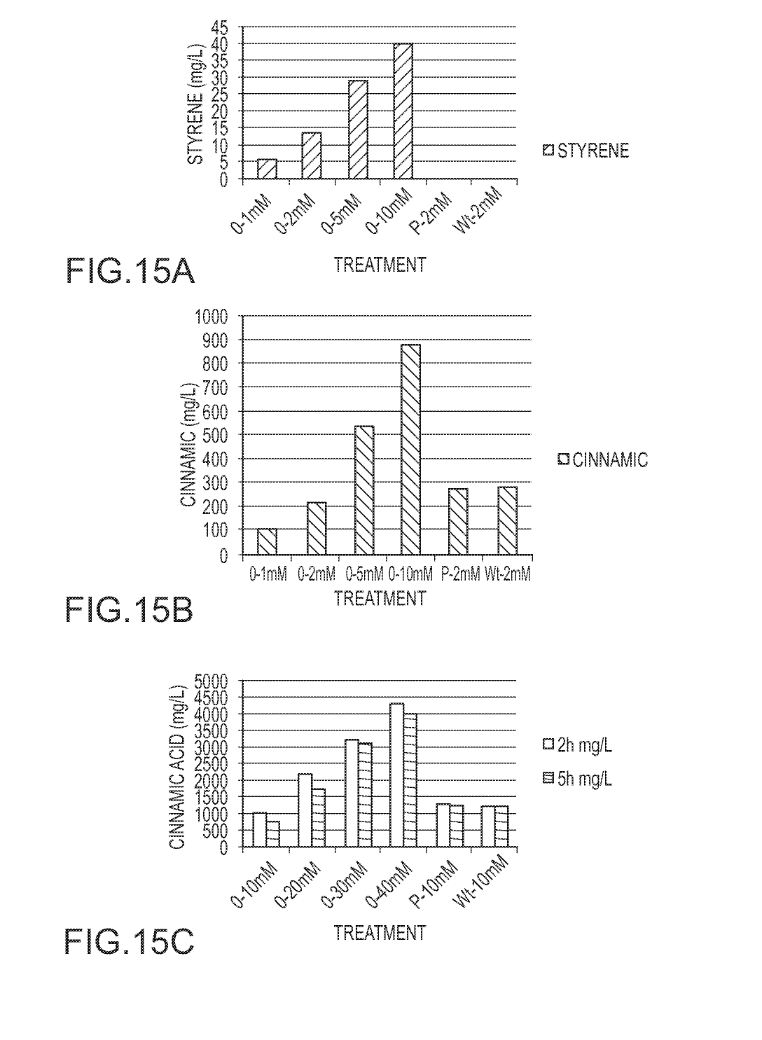

Since the styrene levels kept rising after adding 10 mM of cinnamic acid, higher concentrations were also tested. The highest styrene level (74.3 mg/L) was found in the medium containing OHBA1-transgenic cells fed with 40 mM substrate. The corresponding reduction of substrate was also observed (FIG. 15). These results further show that E. coli expressing PAD1 does not produce styrene.

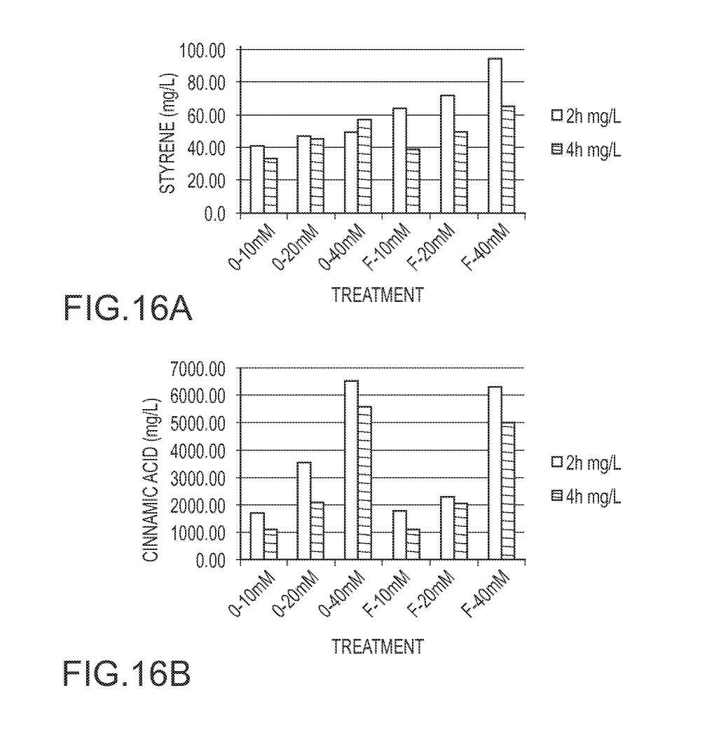

Next, two E. coli samples containing either OHBA1 or FDC were compared under three different feeding concentrations. As compared to OHBA1, FDC-transgenic cells appeared more productive for styrene biosynthesis under the same conditions, 94.6 mg/L vs. 49.6 mg/L after feeding on cinnamic acid substrate for 2 h (FIG. 16).

### Example 12

### Fermentation Production of Styrene

To study the production of styrene under the fermentation conditions, we used the transgenic yeast to carry out a fermentation production of styrene. S. cerevisiae WAT11/pAG305-PAL2/pAG303-FDC was grown in a defined medium (SD-drop out, 1.4 g/L; Ura, 40 mg/L; Adenine, 40 mg/L; YNB, 1.7 g/L; (NH4)2SO4, 5 g/L; and Glucose, 20 g/L). 3 L medium was prepared in a 7 L New Brunswick Scientific fermenter and autoclaved at 121 C for 20 minutes. After autoclave, medium pH turns to 4.20.

Overnight S. cerevisiae OD600 was about 5-6 in the morning. At 11:00 am, the above overnight culture was sub-cultured at 5% into 100 mL fresh medium and incubates at 30° C., 200 rpm in a Brunswick Scientific shaker. At 4:00 pm, 100 mL exponential phase culture was inoculated into fermenter.

The fermenter was maintained at temperature 30° C., air rate 1 VVM and DO maintained at above 30% by rpm control. The outlet gas was cooled by 10° C. cooling water through condenser then goes to 800 mL butanol solution.

24 hour after inoculation, 500 mL 30 g/L phenylalanine was fed to the fermenter (final concentration about 5 g/L) and product was assayed by HPLC.

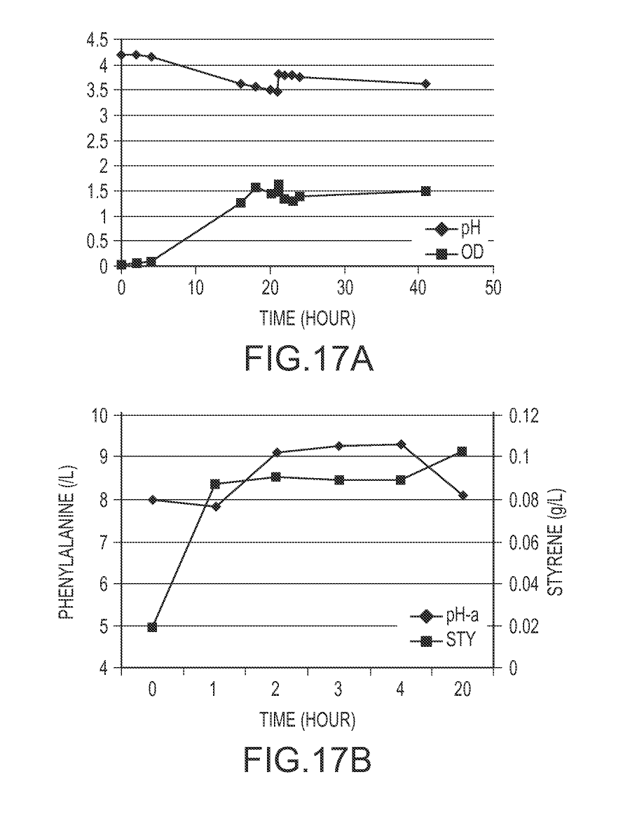

Within two hours, styrene accumulated to about saturation amount in medium phase. However, the styrene did not accumulate much in butanol, which was about 6.7 mg/L after 20 hour phenylalanine feeding (FIG. 17).

### Example 13

### Expression of Yeast FDC1 in E. coli

To examine the activity of purified FDC1 in vitro, we constructed an expression vector and expressed FDC1 in E. coli under various conditions. Plasmid pDEST17-FDC1 was transformed into E. Coli strain BL21(DE3) competent cells. Terrific-broth media supplemented with ampicillin (100 μg/mL) was inoculated with a 1000-time dilution of an overnight culture. The bacteria were cultured at 37° C. until OD₆₀₀ reached 0.8 to 1.0, at which point isopropyl f3-D-1-thiogalactopyranoside (IPTG) was added to a final concentration of 0.2 mM, and continued to be cultured for 3 hours at the same temperature. We used three different lysis buffers to check the expression of FDC1. Buffer A contained 25 mM potassium phosphate buffer pH 7.5, 500 mM sodium chloride, 10 mM imidazole, 20 mM PME, 0.5 mM PMSF, 10 mM MgCl₂, 2 μg/mL DNAse, 2 μg/mL RNAse, and 4 μg/mL lysozyme. Buffer B contained 50 mM potassium phosphate pH buffer 7.0, 1 mM DTT, 50 mM sodium thiosulfate, 20% glycerol, 500 mM NaCl, 10 mM MgCl₂, 20 mM imidazole, 2 μg/mL DNAse, 2 μg/mL RNAse, and 4 μg/mL lysozyme. Buffer C was composed of Buffer A containing 50 mM sodium thiosulfate and 20% glycerol.

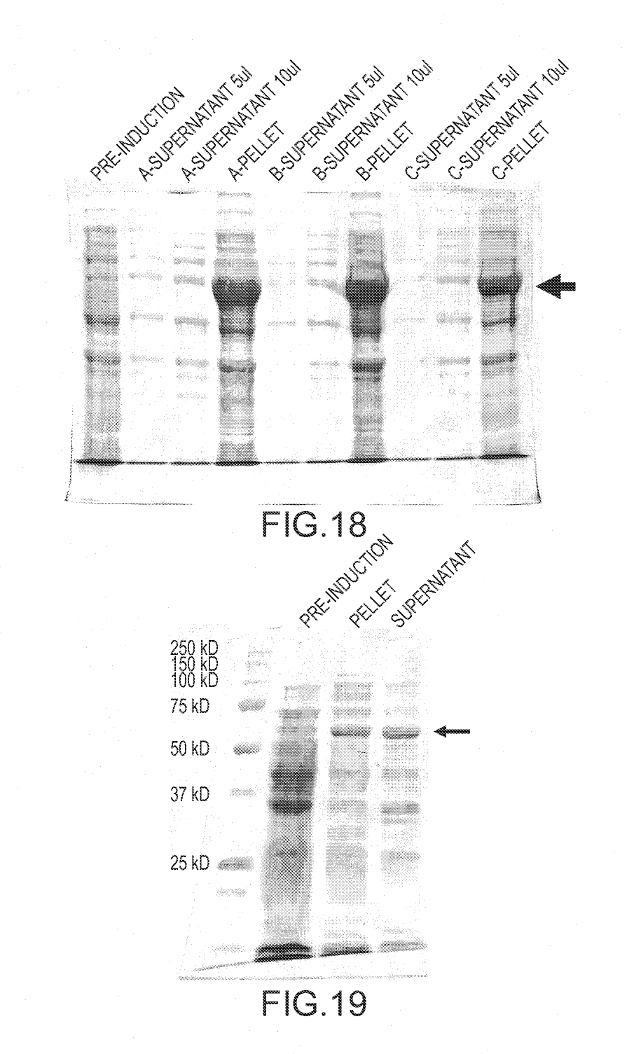

At 37° C., the recombinant FDC1 was not expressed as a soluble form in E. coli, and formed inclusion bodies, as shown in FIG. 18. The standard protocols for expressing recombinant proteins are not applicable for FDC1.

To produce functional FDC1 in E. coli, expression of FDC1 was induced at various temperatures (37° C., 30° C., 25° C., 18° C., and 16° C.) in the presence of various concentrations of IPTG (0.2 mM, 0.5 mM). Concentrations of IPTG had no significant effect on expression; however, lower temperature improved expression and solubility of this protein. Optimal FDC1 expression was achieved after induction with 0.2 mM IPTG at 16° C. for 16 hours (FIG. 19). The cells were harvested by centrifugation at 4500 rpm at 4° C. and washed once with 1×PBS buffer and then stored at −80° C.

Expression of FDC1 was examined by SDS-PAGE (10% acrylamide slab gel, 0.75 mm thick), using the Laemmli protocol. Coomassie brilliant blue R-250 was used to stain the protein band. Our results indicated that expression of FDC1 at lower temperature with lower concentration of IPTG played a role for the correct folding of the enzyme, and the conversion of aggregated or misfolded forms to a soluble, functional form.

### Example 14

### Effect of Metal Ions on FDC1 Activity

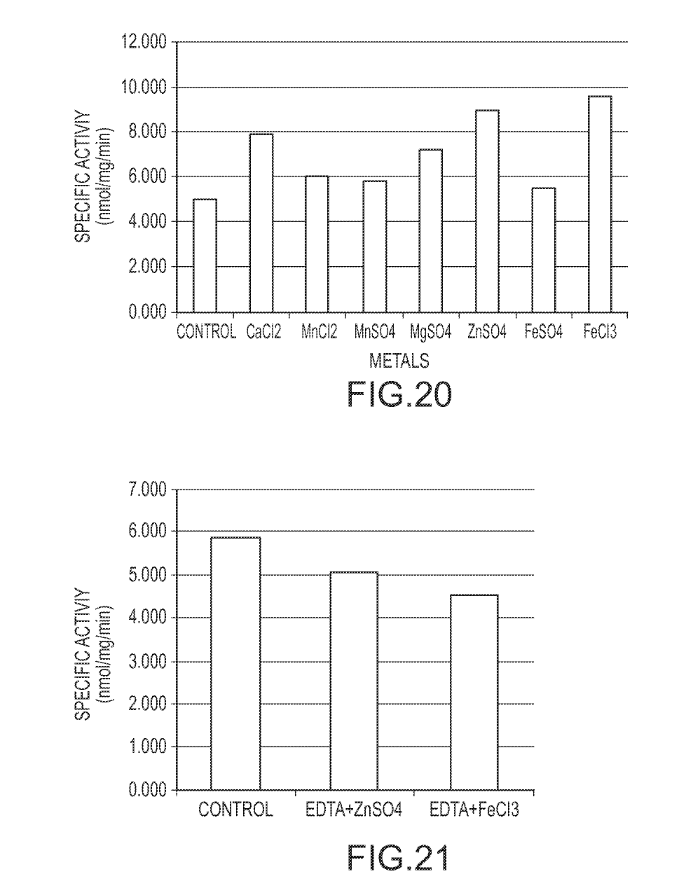

To study the effect of metal ions on FDC1 activity, we carried out reactions with various metal ions, including ZnSO₄, FeCl₃, MnCl₂, MgSO₄, CaCl₂, MnSO₄, and FeSO₄. Reaction mixtures of 25 mM potassium phosphate buffer pH 6.5 containing 1.4 mM tCA, 5 mM DTT, 10 mM metal ion, and 0.5 mg purified FDC1 adjusted to a final volume of 1 mL were incubated for 5 minutes at 30° C. After the reaction, 1 mL of propanol was added to the reaction mixtures to solubilize styrene. The experimental control used the same conditions except the reaction mixture contained no metal ions. The experimental negative control used the same conditions except the reaction mixture contained no substrate. Ca⁺², Mg⁺², Zn⁺², and Fe⁺³ ions increased the activity of FDC1 (FIG. 20).

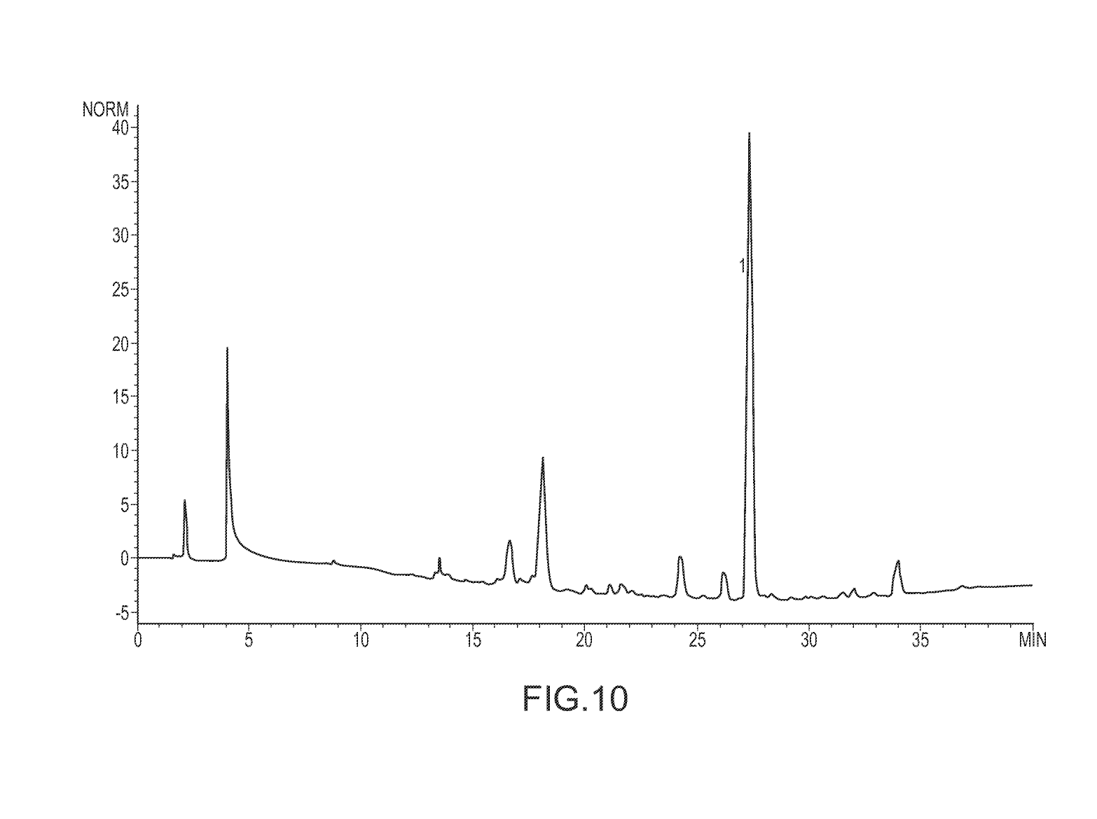

The effects of Zn²⁺ and Fe³⁺ on FDC1 activity were further investigated using EDTA to cancel out the effects of the metal ions. Reaction mixtures of 25 mM potassium phosphate buffer pH 6.5 containing 1.4 mM tCA, 5 mM DTT, 10 mM metal ion, 10 mM EDTA and 0.5 mg purified FDC1 were adjusted to a final volume of 1 mL, and were incubated for 5 minutes at 30° C. After the reaction, 1 mL of propanol was added to the reaction mixtures to solubilize styrene. The experimental control used the same conditions except the reaction mixture contained no metal ions or EDTA. The experimental negative control used the same conditions except the reaction mixture contained no substrate. The reactions containing the metal ion and EDTA had a similar activity as compared to the control (FIG. 21), indicating that Zn²⁺ and Fe³⁺ increased the activity of FDC1. Based on results presented in FIGS. 10A and 10B, enzyme activity of FDC1 can be increased by metal ions and it was not due to experimental artifact. For industrial production of styrene, this piece of information suggests that including certain metal ions in the culture media can be beneficial to styrene biosynthesis.

### Example 15

### Production of Cinnamic Acid from Phenylalanine

Improving or optimizing the conversion of phenylalanine to trans-cinnamic acid can also improve the bioproduction of styrene. As described and exemplified herein, the inventors/inventor also investigated various parameters for trans-cinnamic acid production.

The DNA encoding phenylalanine ammonia lyase (PAL) was transformed in BL21 (DE3) cells and grown in a LB plate containing ampicillin (100 μg/ml) and grown at 37° C. for overnight. One colony was inoculated in 5.0 ml LB containing ampicillin and grown at 30° C. with 250 rpm shaking for overnight. Then 1 ml overnight cultures inoculated 500 ml LB containing ampicillin and were grown at 37° C. with 250 rpm shaking until the OD600 reached 0.8. Induction was carried out by adding 1 mM IPTG and growing for 3 hr at 37° C., then the culture was fed with phenylalanine (2.5 g/L) and continued to culture at 30° C. with shaking at 250 rpm for 12 hr. The strain bearing PAL produced 2.2 g/L of tCA. The amount of tCA was measured by HPLC. The sample (500 μl) was centrifuged at 15000 rpm for 8 min and 400 μl of cleared supernatant was put in a new eppendorf tube and centrifuged again for 15000 rpm for 4 min. Then a 10 μl sample was injected directly to HPLC to measure the amount of tCA.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Although the subject technology has been described with reference to the examples provided above, it should be understood that various modifications can be made without departing from the spirit of the subject technology.

Throughout this application, underlined, italicized and/or boldface headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. A method for the production of styrene comprising:
(i) contacting a recombinant host cell with a fermentable substrate, said recombinant host comprising:
a) at least one polynucleotide encoding a polypeptide having phenylalanine ammonia lyase (PAL) activity; and
b) at least one polynucleotide encoding a polypeptide having cinnamic acid decarboxylase activity; and
(ii) growing said recombinant cell for a time sufficient to produce styrene.

2. The method of claim 1, further comprising recovering said styrene.

3. The method of claim 1, wherein the polypeptide having phenylalanine ammonia lyase (PAL) activity comprises a polypeptide selected from the group consisting of PAL1 as set forth in SEQ ID NO:2, PAL2 as set forth in SEQ ID NO:4, PAL4 as set forth in SEQ ID NO:6, and a functional fragment or variant thereof.

4. The method of claim 1, wherein the polypeptide having cinnamic acid decarboxylase activity comprises a polypeptide selected from the group consisting of ferulic acid decarboxylase (FDC) as set forth in SEQ ID NO:8, AnOHBA1 as set forth in SEQ ID NO:20, and a functional fragment or variant thereof.

5. The method of claim 1, wherein the host cell does not comprise a phenylacrylic acid decarboxylase (PAD1) polynucleotide as set forth in SEQ ID NO:9 or SEQ ID NO:15.

6. The method according to claim 1, wherein said recombinant host cell is selected from the group consisting of bacteria, yeast, filamentous fungi, cyanobacteria algae and plant cells.

7. The method according to claim 1, wherein said recombinant host cell is selected from the group consisting of Escherichia, Salmonella, Bacillus, Acinetobacter, Streptomyces, Corynebacterium, Methylosinus, Methylomonas, Rhodococcus, Pseudomonas, Rhodobacter, Synechocystis; Saccharomyces, Zygosaccharomyces, Kluyveromyces, Candida, Hansenula, Debaryomyces, Mucor, Pichia, Torulopsis, Aspergillus, Arthrobotrys, Brevibacteria, Microbacterium, Arthrobacter, Citrobacter, Escherichia, Klebsiella, Pantoea, Salmonella Corynebacterium, Clostridium, and Clostridium acetobutylicum.

8. The method according to claim 1, wherein said recombinant host cell is a cell isolated from plants selected from the group consisting of soybean, rapeseed, sunflower, cotton, corn, tobacco, alfalfa, wheat, barley, oats, sorghum, rice, broccoli, cauliflower, cabbage, parsnips, melons, carrots, celery, parsley, tomatoes, potatoes, strawberries, peanuts, grapes, grass seed crops, sugar beets, sugar cane, beans, peas, rye, flax, hardwood trees, softwood trees, forage grasses, Arabidopsis thaliana, rice (Oryza sativa), switchgrass (Panicum vigratum), Brachypodium spp, Brassica spp., and Crambe abyssinica.

9. The method according to claim 1, wherein the polynucleotide encoding a polypeptide having phenylalanine ammonia lyase activity is derived from Arabidopsis.

10. The method according to claim 1, wherein the polynucleotide encoding a polypeptide having cinnamic acid decarboxylase activity is derived from S. cerevisiae.

11. The method according to claim 1, wherein the styrene can be further converted to other organic molecules by enzymatic or biological conversion within the host cell.

12. The method according to claim 11, wherein the styrene is further converted to toluene, xylene or polystyrene.

13. A recombinant host cell comprising:
at least one polynucleotide encoding a polypeptide having phenylalanine ammonia lyase (PAL) activity; and
at least one polynucleotideencoding a polypeptide having cinnamic acid decarboxylase activity;
wherein said recombinant host cell is capable of producing styrene when grown under a suitable culture condition.

14. The recombinant host cell of claim 13, wherein the polypeptide having phenylalanine ammonia lyase (PAL) activity comprises a polypeptide selected from the group consisting of PAL1 as set forth in SEQ ID NO:2, PAL2 as set forth in SEQ ID NO:4, PAL4 as set forth in SEQ ID NO:6, and a functional fragment or variant thereof.

15. The recombinant host cell of claim 13, the polypeptide having cinnamic acid decarboxylase activity comprises a polypeptide selected from the group consisting of ferulic acid decarboxylase (FDC) as set forth in SEQ ID NO:8, AnOHBA1 as set forth in SEQ ID NO:20, and a functional fragment or variant thereof.

16. The recombinant host cell of claim 13, wherein the host cell does not comprise a phenylacrylic acid decarboxylase (PAD1) polynucleotide as set forth in SEQ ID NO:9 or SEQ ID NO:15.

17. A method for the production of styrene comprising:
(i) contacting a recombinant host cell with a fermentable substrate, said recombinant host comprising:
a) at least one polynucleotide encoding a polypeptide having phenylalanine ammonia lyase (PAL) activity; and
b) at least one polynucleotide encoding a polypeptide having cinnamic acid decarboxylase activity; and
(ii) growing said recombinant cell for a time sufficient to produce styrene;
wherein the polypeptide having phenylalanine ammonia lyase (PAL) activity comprises a polypeptide of any one of PAL1 as set forth in SEQ ID NO:2, PAL2 as set forth in SEQ ID NO:4, PAL4 as set forth in SEQ ID NO:6, or a functional fragment or variant thereof;
wherein the polypeptide having cinnamic acid decarboxylase activity comprises a polypeptide of any one of ferulic acid decarboxylase (FDC) as set forth in SEQ ID NO:8, AnOHBA1 as set forth in SEQ ID NO:20, or a functional fragment or variant thereof.

18. The method of claim 17, further comprising recovering said styrene.

19. The method of claim 17, wherein the host cell does not comprise a phenylacrylic acid decarboxylase (PAD1) polynucleotide as set forth in SEQ ID NO:9 or SEQ ID NO:15.

20. A recombinant host cell comprising:
at least one polynucleotide encoding a polypeptide having phenylalanine ammonia lyase (PAL) activity; and
at least one polynucleotide encoding a polypeptide having cinnamic acid decarboxylase activity;
wherein said recombinant cell produces styrene when grown under a suitable culture condition;
wherein the polypeptide having phenylalanine ammonia lyase (PAL) activity comprises a polypeptide of any one of PAL1 as set forth in SEQ ID NO:2, PAL2 as set forth in SEQ ID NO:4, PAL4 as set forth in SEQ ID NO:6, or a functional fragment or variant thereof;
wherein the polypeptide having cinnamic acid decarboxylase activity comprises a polypeptide of any one of ferulic acid decarboxylase (FDC) as set forth in SEQ ID NO:8, AnOHBA1 as set forth in SEQ ID NO:20, or a functional fragment or variant thereof.

21. The recombinant host cell of claim 20, wherein the host cell does not contain a phenylacrylic acid decarboxylase (PAD1) gene as set forth in SEQ ID NO:9 or SEQ ID NO:15.
